SEQUENCE LISTING (19 sequences)

SEQ ID NO: 1 - protein, 114 aa, Artificial Sequence
  Description of Artificial Sequence Synthetic construct
DIQLTQSPSSLSASVGDRVTITCRASKPVDGEGDSYMNWYQQKPGKAPKLLIYAASYLESGVPSRFSGSGSGTDFTLTISSLQPEDFATYYCQQSHEDPYTFGQGTKVEIKRTV

SEQ ID NO: 2 - protein, 114 aa, Artificial Sequence
  Description of Artificial Sequence Synthetic construct
DIQLTQSPSSLSASVGDRVTITCRASQSVDYEGDSYMNWYQQKPGKAPKLLIYAASYLESGVPSRFSGSGSGTDFTLTISSLQPEDFATYYCQQSHEDPYTFGQGTKVEIKRTV

SEQ ID NO: 3 - protein, 114 aa, Artificial Sequence
  Description of Artificial Sequence Synthetic construct
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTV

SEQ ID NO: 4 - protein, 114 aa, Artificial Sequence
  Description of Artificial Sequence Synthetic construct
EVQLVESGGGLVQPGGSLRLSCAVSGYSITSGYSWNWIRQAPGKGLEWVASIKYSGETKYNPSVKGRITISRDDSKNTFYLQMNSLRAEDTAVYYCARGSHYFGHWHFAVWGQG

SEQ ID NO: 5 - protein, 114 aa, Artificial Sequence
  Description of Artificial Sequence Synthetic construct
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQG

SEQ ID NO: 6 - protein, 218 aa, Artificial Sequence
  Description of Artificial Sequence Synthetic construct
DIQLTQSPSSLSASVGDRVTITCRASQSVDYDGDSYMNWYQQKPGKAPCLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 451 aa, Artificial Sequence
  Description of Artificial Sequence Synthetic construct
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 218 aa, Artificial Sequence
  Description of Artificial Sequence Synthetic construct
DIQLTQSPSSLSASVGDRVTITCRASKPVDGEGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 451 aa, Artificial Sequence; identical to SEQ ID NO: 7
  Description of Artificial Sequence Synthetic construct

SEQ ID NO: 10 - protein, 218 aa, Artificial Sequence; identical to SEQ ID NO: 8
  Description of Artificial Sequence Synthetic construct

SEQ ID NO: 11 - protein, 451 aa, Artificial Sequence
  Description of Artificial Sequence Synthetic construct
EVQLVESGGGLVQPGGSLRLSCAVSGYSITSGYSWNWIRQAPGKGLEWVASIKYSGETKYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 218 aa, Artificial Sequence
  Description of Artificial Sequence Synthetic construct
DIQLTQSPSSLSASVGDRVTITCRASKPVDGEGDSYLNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 229 aa, Artificial Sequence
  Description of Artificial Sequence Synthetic construct
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 14 - protein, 229 aa, Artificial Sequence
  Description of Artificial Sequence Synthetic construct
EVQLVESGGGLVQPGGSLRLSCAVSGYSITSGYSWNWIRQAPGKGLEWVASIKYSGETKYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 15 - protein, 248 aa, Artificial Sequence
  Description of Artificial Sequence Synthetic construct
EVQLVESGGGLVQPGGSLRLSCAVSGYSITSGYSMNWIRQAPGKGLEWVASITYDGSTNYNPSVKGRITISKDDSKNTFYLQMNSLRAEDTAVYYCARGSHYFGHWHFAVWGQGTLVTVSSEGGGSEGGGSEGGGSDIQLTQSPSSLSASVGDRVTITCRASKPVDGEGDSYLNWYQQKPGKAPKLLIYAASYLESGVPSRFSGSGSGTDFTLTISSLQPEDFATYYCQQSHEDPYTFGQGTKVEIKR

SEQ ID NO: 16 - protein, 248 aa, Artificial Sequence
  Description of Artificial Sequence Synthetic construct
EVQLVESGGGLVQPGGSLRLSCAVSGYSITSGYSMNWIRQAPGKGLEWVASIKYSGETKYNPSVKGRITISKDDSKNTFYLQMNSLRAEDTAVYYCARGSHYFGHWHFAVWGQGTLVTVSSEGGGSEGGGSEGGGSDIQLTQSPSSLSASVGDRVTITCRASKPVDGEGDSYLNWYQQKPGKAPKLLIYAASYLESGVPSRFSGSGSGTDFTLTISSLQPEDFATYYCQQSHEDPYTFGQGTKVEIKR

SEQ ID NO: 17 - protein, 218 aa, Artificial Sequence; identical to SEQ ID NO: 12
  Description of Artificial Sequence Synthetic construct

SEQ ID NO: 18 - protein, 233 aa, Artificial Sequence
  Description of Artificial Sequence Synthetic construct
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPC

SEQ ID NO: 19 - protein, 233 aa, Artificial Sequence
  Description of Artificial Sequence Synthetic construct
EVQLVESGGGLVQPGGSLRLSCAVSGYSITSGYSWNWIRQAPGKGLEWVASIKYSGETKYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPC

(19) United States
(12) Patent Application Publication
(10) Pub. No.: US 2005/0261208 A1
(43) Pub. Date: Nov. 24, 2005

(54) Combination of dehydroepiandrosterone or dehydroepiandrosterone-sulfate with an anti-IgE antibody for treatment of asthma or chronic obstructive pulmonary disease

(76) Inventors: Cynthia B. Robinson, Wayne, PA (US); Howard A. Ball, Kendall Park, NJ (US)

Correspondence Address: WILSON SONSINI GOODRICH & ROSATI, 650 PAGE MILL ROAD, PALO ALTO, CA, 94304-1050, US

(21) Appl. No.: 10/923,327

(22) Filed: Aug. 20, 2004

Related U.S. Application Data
(63) Continuation-in-part of application No. 10/698,073, filed on Oct. 29, 2003.
(63) Continuation-in-part of application No. PCT/US04/25054, filed on Jul. 30, 2004.
(60) Provisional application No. 60/492,231, filed on Jul. 31, 2003.

(51) Int. Cl. A61K 31/704; A61K 31/58; A61K 31/56

(52) U.S. Cl. 514/25; 514/169; 514/172

(57) ABSTRACT

A pharmaceutical or veterinary composition, comprises a first active agent selected from a dehydroepiandrosterone and/or dehydroepiandrosterone-sulfate, or a salt thereof, and a second active agent comprising an anti-IgE antibody for the treatment of asthma, chronic obstructive pulmonary disease, or other respiratory diseases. The composition is provided in various formulations and in the form of a kit. The products of this patent are applied to the prophylaxis and treatment of asthma, chronic obstructive pulmonary disease, or other respiratory diseases.

### REFERENCE TO RELATED APPLICATIONS

This application is a continuation-in-part of U.S. application Ser. No. 10/698,073, filed on Oct. 29, 2003 and PCT application Ser. No. ______, filed on Jul. 30, 2004, which claim priority to the U.S. Provisional Patent Application Ser. No. 60/492,231, filed on Jul. 31, 2003, which are herein incorporated by reference in their entirety.

### BACKGROUND OF THE INVENTION

1. Field of the Invention

This invention relates to a composition comprising a non-glucocorticoid steroid including dehydroepiandrosterone (DHEA), DHEA-Sulfate, or a salt thereof, and an anti-IgE antibody. These compositions are useful in the treatment of asthma, chronic obstructive pulmonary disease (COPD), or other respiratory diseases.

2. Description of the Background

Respiratory ailments, associated with a variety of conditions, are extremely common in the general population. In some cases they are accompanied by inflammation, which aggravates the condition of the lungs. Respiratory ailments include asthma, chronic obstructive pulmonary disease (COPD), and other upper and lower airway respiratory diseases, such as, allergic rhinitis, Acute Respiratory Distress Syndrome (ARDS), and pulmonary fibrosis.

Asthma, for example, is one of the most common diseases in industrialized countries. In the United States it accounts for about 1% of all health care costs. An alarming increase in both the prevalence and mortality of asthma over the past decade has been reported, and asthma is predicted to be the preeminent occupational lung disease in the next decade. Asthma is a condition characterized by variable, in many instances reversible obstruction of the airways. This process is associated with lung inflammation and in some cases lung allergies. Many patients have acute episodes referred to as “asthma attacks,” while others are afflicted with a chronic condition. The asthmatic process is believed to be triggered in some cases by inhalation of antigens by hypersensitive subjects. This condition is generally referred to as “extrinsic asthma.” Other asthmatics have an intrinsic predisposition to the condition, which is thus referred to as “intrinsic asthma,” and may be comprised of conditions of different origin, including those mediated by the adenosine receptor(s), allergic conditions mediated by an immune IgE-mediated response, and others. All asthmatics have a group of symptoms, which are characteristic of this condition: episodic bronchoconstriction, lung inflammation and decreased lung surfactant. Existing bronchodilators and anti-inflammatories are currently commercially available and are prescribed for the treatment of asthma. The most common anti-inflammatories, corticosteroids, have considerable side effects but are commonly prescribed nevertheless. Most of the drugs available for the treatment of asthma are, more importantly, barely effective in a number of patients.

COPD is characterized by airflow obstruction that is generally caused by chronic bronchitis, emphysema, or both. Commonly, the airway obstruction is incompletely reversible but 10-20% of patients do show some improvement in airway obstruction with treatment. In chronic bronchitis, airway obstruction results from chronic and excessive secretion of abnormal airway mucus, inflammation, bronchospasm, and infection. Chronic bronchitis is also characterized by chronic cough, mucus production, or both, for at least three months in at least two successive years where other causes of chronic cough have been excluded. In emphysema, a structural element (elastin) in the terminal bronchioles is destroyed leading to the collapse of the airway walls and inability to exhale “stale” air. In emphysema there is permanent destruction of the alveoli. Emphysema is characterized by abnormal permanent enlargement of the air spaces distal to the terminal bronchioles, accompanied by destruction of their walls and without obvious fibrosis. COPD can also give rise to secondary pulmonary hypertension. Secondary pulmonary hypertension itself is a disorder in which blood pressure in the pulmonary arteries is abnormally high. In severe cases, the right side of the heart must work harder than usual to pump blood against the high pressure. If this continues for a long period, the right heart enlarges and functions poorly, and fluid collects in the ankles (edema) and belly. Eventually the left heart begins to fail. Heart failure caused by pulmonary disease is called cor pulmonale.

COPD characteristically affects middle aged and elderly people, and is one of the leading causes of morbidity and mortality worldwide. In the United States it affects about 14 million people and is the fourth leading cause of death, and the third leading cause for disability in the United States. Both morbidity and mortality, however, are rising. The estimated prevalence of this disease in the United States has risen by 41% since 1982, and age adjusted death rates rose by 71% between 1966 and 1985. This contrasts with the decline over the same period in age-adjusted mortality from all causes (which fell by 22%), and from cardiovascular diseases (which fell by 45%). In 1998 COPD accounted for 112,584 deaths in the United States.

COPD, however, is preventable, since it is believed that its main cause is exposure to cigarette smoke. Long-term smoking is the most frequent cause of COPD. It accounts for 80 to 90% of all cases. A smoker is 10 times more likely than a non-smoker to die of COPD. The disease is rare in lifetime non-smokers, in whom exposure to environmental tobacco smoke will explain at least some of the airways obstruction. Other proposed etiological factors include airway hyper responsiveness or hypersensitivity, ambient air pollution, and allergy. The airflow obstruction in COPD is usually progressive in people who continue to smoke. This results in early disability and shortened survial time. Smoking cessation shows the rate of decline to that of a non-smoker but the damage caused by smoking is irreversible. Other risk factors include: heredity, second-hand smoke, exposure to air pollution at work and in the environment, and a history of childhood respiratory infections. The symptoms of COPD include: chronic coughing, chest tightness, shortness of breath at rest and during exertion, an increased effort to breathe, increased mucus production, and frequent clearing of the throat.

There is very little currently available to alleviate symptoms of COPD, prevent exacerbations, preserve optimal lung function, and improve daily living activities and quality of life. Many patients will use medication chronically for the rest of their lives, with the need for increased doses and additional drugs during exacerbations. Medications that are currently prescribed for COPD patients include: fast-acting β2-agonists, anticholinergic bronchodilators, long-acting bronchodilators, antibiotics, and expectorants. Amongst the currently available treatments for COPD, short term benefits, but not long term effects, were found on its progression, from administration of anti-cholinergic drugs, β2 adrenergic agonists, and oral steroids. Oral steroids are only recommended for acute exacerbations with long term use contributing to excess mortality and morbidity.

Short and long acting inhaled β2 adrenergic agonists achieve short-term bronchodilation and provide some symptomatic relief in COPD patients, but show no meaningfuil maintenance effect on the progression of the disease. Short acting β2 adrenergic agonists improve symptoms in subjects with COPD, such as increasing exercise capacity and produce some degree of bronchodilation, and even an increase in lung function in some severe cases. The maximum effectiveness of the newer long acting inhaled, β2 adrenergic agonists was found to be comparable to that of short acting β2 adrenergic agonists. Salmeterol was found to improve symptoms and quality of life, although only producing modest or no change in lung function. The use of β2-agonists can produce cardiovascular effects, such as altered pulse rate, blood pressure and electrocardiogram results. In rare cases, the use of β2-agonists can produce hypersensitivity reactions, such as urticaria, angioedema, rash and oropharyngeal edema. In these cases, the use of the β2-agonist should be discontinued. Continuous treatment of asthmatic and COPD patients with the bronchodilators ipratropium bromide or fenoterol was not superior to treatment on an as-needed basis, therefore indicating that they are not suitable for maintenance treatment. The most common immediate adverse effect of β2 adrenergic agonists, on the other hand, is tremors, which at high doses may cause a fall in plasma potassium, dysrhythmias, and reduced arterial oxygen tension. The combination of a β2 adrenergic agonist with an anti-cholinergic drug provides little additional bronchodilation compared with either drug alone. The addition of ipratropium to a standard dose of inhaled β2 adrenergic agonists for about 90 days, however, produces some improvement in stable COPD patients over either drug alone. Overall, the occurrence of adverse effects with β2 adrenergic agonists, such as tremor and dysrhythmias, is more frequent than with anti-cholinergics. Thus, neither anti-cholinergic drugs nor β2 adrenergic agonists have an effect on all people with COPD; nor do the two agents combined.

Anti-cholinergic drugs achieve short-term bronchodilation and produce some symptom relief in people with COPD, but no improved long-term prognosis. Most COPD patients have at least some measure of airways obstruction that is somewhat alleviated by ipratropium bromide. “The Lung Health Study” found spirometric signs of early COPD in men and women smokers and followed them for five years. Three treatments were compared over a five year period and results show that ipratropium bromide had no significant effect on the decline in the functional effective volume of the patient's lungs whereas smoking cessation produced a slowing of the decline in the functional effective volume of the lungs. Ipratropium bromide, however, produced adverse effects, such as cardiac symptoms, hypertension, skin rashes, and urinary retention.

Theophyllines produce modest bronchodilation in COPD patients whereas they have frequent adverse effects, and a small therapeutic range. Serum concentrations of 15-20 mg/l are required for optimal effects and serum levels must be carefully monitored. Adverse effects include nausea, diarrhea, headache, irritability, seizures, and cardiac arrhythmias, occurring at highly variable blood concentrations and, in many people, even within the therapeutic range. The theophyllines' doses must be adjusted individually according to smoking habits, infection, and other treatments, which is cumbersome. Although theophyllines have been claimed to have an anti-inflammatory effect in asthma, especially at lower doses, none has been reported in COPD. The adverse effects of theophyllines and the need for frequent monitoring limit their usefulness.

Oral corticosteroids have been shown to improve the short term outcome in acute exacerbations of COPD but long term administration of oral steroid has been associated with serious side effects including osteoporosis and inducing overt diabetes. Inhaled corticosteroids have been found to have no real short-term effect on airway hyper-responsiveness to histamine. In two studies of 3 year treatment with inhaled fluticasone, moderate and severe exacerbations were significantly reduced as well as a modest improvement in the quality of life without affecting pulmonary function. COPD patients with more reversible disease seem to benefit more from treatment with inhaled fluticasone.

Mucolytics have a modest beneficial effect on the frequency and duration of exacerbations but an adverse effect on lung function. Neither N-acetylcysteine nor other mucolytics, however, have a significant effect in people with severe COPD (functional effective volume<50%) in spite of evidencing greater reductions in frequency of exacerbation. N-acetylcysteine produced gastrointestinal side effects. Long-term oxygen therapy administered to hypoxaemic COPD and congestive cardiac failure patients, had little effect on their rates of death for the first 500 days or so, but survival rates in men increased afterwards and remained constant over the next five years. In women, however, oxygen decreased the rates of death throughout the study. Continuous oxygen treatment of hypoxemic COPD patients for 19.3 years decreased overall risk of death. To date, however, only life style changes, smoking cessation and long term treatment with oxygen (in hypoxaemics), have been found to alter the long-term course of COPD.

Antibiotics are also often given at the first sign of a respiratory infection to prevent farther damage and infection in diseased lungs. Expectorants help loosen and expel mucus secretions from the airways, and may help make breathing easier. In addition, other medications may be prescribed to manage conditions associated with COPD. These may include: diuretics (which are given as therapy to avoid excess water retention associated with right-heart failure), digitalis (which strengthens the force of the heartbeat), and cough suppressants. This latter list of medications help alleviate symptoms associated with COPD but do not treat COPD. Thus, there is very little currently available to alleviate symptoms of COPD, prevent exacerbations, preserve optimal lung function, and improve daily living activities and quality of life.

Acute Respiratory Distress Syndrome (ARDS), or stiff lung, shock lung, pump lung and congestive atelectasis, is believed to be caused by fluid accumulation within the lung which, in turn, causes the lung to stiffen. The condition is triggered within 48 hours by a variety of processes that injure the lungs such as trauma, head injury, shock, sepsis, multiple blood transfusions, medications, pulmonary embolism, severe pneumonia, smoke inhalation, radiation, high altitude, near drowning, and others. In general, ARDS occurs as a medical emergency and may be caused by other conditions that directly or indirectly cause the blood vessels to “leak” fluid into the lungs. In ARDS, the ability of the lungs to expand is severely decreased and produces extensive damage to the air sacs and lining or endothelium of the lung. ARDS' most common symptoms are labored, rapid breathing, nasal flaring, cyanosis blue skin, lips and nails caused by lack of oxygen to the tissues, anxiety, and temporarily absent breathing. A preliminary diagnosis of ARDS may be confirmed with chest X-rays and the measurement of arterial blood gas. In some cases ARDS appears to be associated with other diseases, such as acute myelogenous leukemia, with acute tumor lysis syndrome (ATLS) developed after treatment with, e.g. cytosine arabinoside. In general, however, ARDS appears to be associated with traumatic injury, severe blood infections such as sepsis, or other systemic illness, high dose radiation therapy and chemotherapy, and inflammatory responses which lead to multiple organ failure, and in many cases death. In premature babies (“preemies”), neither the lung tissue nor the surfactant is fully developed. When Respiratory Distress Syndrome (RDS) occurs in preemies, it is an extremely serious problem. Preterm infants exhibiting RDS are currently treated by ventilation and administration of oxygen and surfactant preparations. When preemies survive RDS, they frequently develop bronchopulmonary dysplasia (BPD), also called chronic lung disease of early infancy, which is often fatal.

Allergic rhinitis afflicts one in five Americans, accounting for an estimated $4 to 10 billion in health care costs each year, and occurs at all ages. Because many people mislabel their symptoms as persistent colds or sinus problems, allergic rhinitis is probably underdiagnosed. Typically, IgE combines with allergens in the nose to produce chemical mediators, induction of cellular processes, and neurogenic stimulation, causing an underlying inflammation. Symptoms include ocular and nasal congestion, discharge, sneezing, and itching. Over time, allergic rhinitis sufferers often develop sinusitis, otitis media with effusion, and nasal polyposis. Approximately 60% of patients with allergic rhinitis also have asthma and flares of allergic rhinitis aggravate asthma. Degranulation of mast cells results in the release of preformed mediators that interact with various cells, blood vessels, and mucous glands to produce the typical rhinitis symptoms. Most early- and late-phase reactions occur in the nose after allergen exposure. The late-phase reaction is seen in chronic allergic rhinitis, with hypersecretion and congestion as the most prominent symptoms. Repeated exposure causes a hypersensitivity reaction to one or many allergens. Sufferers may also become hyperreactive to nonspecific triggers such as cold air or strong odors. Nonallergic rhinitis may be induced by infections, such as viruses, or associated with nasal polyps, as occurs in patients with aspirin idiosyncrasy.

Medical conditions such as pregnancy or hypothyroidism and exposure to occupational factors or medications may cause rhinitis. The so-called NARES syndrome (Nonallergic Rhinitis with Eosinophilia Syndrome) is a non-allergic type of rhinitis associated with eosinophils in the nasal secretions, which typically occurs in middle-age and is accompanied by some loss of sense of smell. Treatment of allergic and non-allergic rhinitis is unsatisfactory. Self-administered saline improves nasal stuffiness, sneezing, and congestion and usually causes no side effects and it is, thus, the first treatment tried in pregnant patients. Saline sprays are generally used to relieve mucosal irritation or dryness associated with various nasal conditions, minimize mucosal atrophy, and dislodge encrusted or thickened mucus. If used immediately before intranasal corticosteroid dosing, saline sprays may help prevent drug-induced local irritation. Anti-histamines such as terfenadine and astemizole are also employed to treat allergic rhinitis; however, use of antihistamines have been associated with a ventricular arrhythmia known as Torsades de Points, usually in interaction with other medications such as ketoconazole and erythromycin, or secondary to an underlying cardiac problem. Loratadine, another non-sedating anti-histamine, and cetirizine have not been associated with an adverse impact on the QT interval, or with serious adverse cardiovascular events. Cetirizine, however, produces extreme drowsiness and has not been widely prescribed. Non-sedating anti-histamines, e.g. Claritin, may produce some relieving of sneezing, runny nose, and nasal, ocular and palatal itching, but have not been tested for asthma or other more specific conditions. Terfenadine, loratadine and astemizole, on the other hand, exhibit extremely modest bronchodilating effects, reduction of bronchial hyper-reactivity to histamine, and protection against exercise- and antigen-induced bronchospasm. Some of these benefits, however, require higher-than-currently-recommended doses. The sedating-type anti-histamines help induce night sleep, but they cause sleepiness and compromise performance if taken during the day. When employed, anti-histamines are typically combined with a decongestant to help relieve nasal congestion. Sympathomimetic medications are used as vasoconstrictors and decongestants. The three commonly prescribed systemic decongestants, pseudoephedrine, phenylpropanolamine and phenylephrine cause hypertension, palpitations, tachycardia, restlessness, insomnia and headache. The interaction of phenylpropanolamine with caffeine, in doses of two to three cups of coffee, may significantly raise blood pressure. In addition, medications such as pseudoephedrine may cause hyperactivity in children. Topical decongestants, nevertheless, are only indicated for a limited period of time, as they are associated with a rebound nasal dilatation with overuse. Anti-cholinergic agents are given to patients with significant rhinorrhea or for specific conditions such as “gustatory rhinitis”, usually caused by ingestion of spicy foods, and may have some beneficial effects on the common cold. Cromolyn, for example, if used prophylactically as a nasal spray, reduces sneezing, rhinorrhea, and nasal pruritus, and blocks both early- and late-phase hypersensitivity responses, but produces sneezing, transient headache, and even nasal burning. Topical corticosteroids such as Vancenase are effective in the treatment of rhinitis, especially for symptoms of itching, sneezing, and runny nose but are less effective against nasal stuffiness. Depending on the preparation, however, corticosteroid nose sprays may cause irritation, stinging, burning, or sneezing, as well. Local bleeding and septal perforation can also occur sometimes, especially if the aerosol is not aimed properly. Topical steroids generally are more effective than cromolyn sodium in the treatment of allergic rhinitis. Imimunotherapy, while expensive and inconvenient, often provides benefits, especially for inpatients who experience side effects from other medications. So-called blocking antibodies, and agents that alter cellular histamine release, eventually result in decreased IgE, along with many other favorable physiologic changes. This effect is useful in IgE-mediated diseases, e.g., hypersensitivity in atopic patients with recurrent middle ear infections.

Pulmonary fibrosis, interstitial lung disease (ILD), or interstitial pulmonary fibrosis, include more than 130 chronic lung disorders that affect the lung by damaging lung tissue, and produce inflammation in the walls of the air sacs in the lung, scarring or fibrosis in the interstitium (or tissue between the air sacs), and stiffening of the lung. Breathlessness during exercise may be one of the first symptoms of these diseases, and a dry cough may be present. Neither the symptoms nor X-rays are often sufficient to differentiate various types of pulmonary fibrosis. Some pulmonary fibrosis patients have known causes and some have unknown or idiopathic causes. The course of this disease is generally unpredictable and the disease is inevitably fatal. Its progression includes thickening and stiffening of the lung tissue, inflammation and difficult breathing. Most people may need oxygen therapy and the only treatment is lung transplantation.

Lung cancer is the most common cancer in the world. During 2003, there will be about 171,900 new cases of lung cancer (91,800 among men and 80, 100 among women) in the US alone and approximiately 375,000 cases in Europe. Lung cancer is the leading cause of cancer death among both men and women. There will be an estimated 157,200 deaths from lung cancer (88,400 among men and 68,800 among women) in 2003, accounting for 28% of all cancer deaths in the US alone. More people die of lung cancer than of colon, breast, and prostate cancers combined (American Cancer Society Web site, 2003, Detailed Guide: Lung Cancer: What are the Key Statistics?). Tobacco smoking is well established as the main cause of lung cancer and about 90% of cases are thought to be tobacco related. There is a clear dose-response relation between lung-cancer risk and the number of cigarettes smoked per day, degree of inhalation, and age at initiation of smoking. Lifelong smokers have a lung-cancer risk 20-30 times greater than a non-smoker. However, risk of lung cancer decreases with time since smoking cessation. The relative risk of male ex-smokers decreases strongly with time since end of exposure, but does not reach the risk of non-smokers, and does not decrease as much as for female ex-smokers (Tyczynski et al., Lancet Oncol. 4(1):45-55 (2003).

Frequently, COPD and lung cancer are co-morbid diseases and the degree of underlying COPD may dictate whether a particular patient is a surgical candidate. For NSCLC (non small cell lung cancer), only surgery (with or without radiation therapy or adjuvant chemotherapy) is curative.
The 1-year survival rate (the number of people who live at least 1 year after their cancer is diagnosed) for lung cancer was 42% in 1998, largely due to improvements in surgical techniques.
The 5-year survival rate for all stages of non-small cell lung cancer combined is only 15%. For small cell lung cancer the 5-year relative survival rate is about 6%.
For people whose NSCLC is found and treated early with surgery, before it has spread to lymph nodes or other organs, the average 5-year survival rate is about 50%. However, only 15% of people with lung cancer are diagnosed at this early, localized stage.

Clearly, there is much room for improvement in chemoprophylaxis of lung cancer as well as treatment of lung cancer.

Dehydroepiandrosterone (DHEA) (3β-hydroxyandrost-5-en-17-one) is a naturally occurring steroid secreted by the adrenal cortex with apparent chemoprotective properties. Epidemiological studies have shown that low endogenous levels of DHEA correlate with increased risk of developing some forms of cancer, such as pre-menopausal breast cancer in women and bladder cancer in both sexes. The ability of DHEA and DHEA analogues, such as DHEA-S (DHEA-sulfate), to inhibit carcinogenesis is believed to result from their uncompetitive inhibition of the activity of the enzyme glucose-6-phosphate dehydrogenase (G6PDH). G6PDH is the rate limiting enzyme of the hexose monophosphate pathway, a major source of intracellular ribose-5-phosphate and NADPH. Ribose-5-phosphate is a necessary substrate for the synthesis of both ribo- and deoxyribonucleotides. NADPH is a cofactor also involved in nucleic acid biosynthesis and the synthesis of hydroxmethylglutaryl Coenzyme A reductase (HMG CoA reductase). HMG CoA reductase is an unusual enzyme that requires two moles of NADPH for each mole of product, mevalonate, produced. Thus, it appears that HMG CoA reductase would be ultrasensitive to DHEA-mediated NADPH depletion, and that DHEA-treated cells would rapidly show the depletion of intracellular pools of mevalonate. Mevalonate is required for DNA synthesis, and DHEA arrests human cells in the G1 phase of the cell cycle in a manner closely resembling that of the direct HMG CoA. Because G6PDH is required to produces mevalonic acid used in cellular processes such as protein isoprenylation and the synthesis of dolichol, a precursor for glycoprotein biosynthesis, DHEA inhibits carcinogenesis by depleting mevalonic acid and thereby inhibiting protein isoprenylation and glycoprotein synthesis. Mevalonate is the central precursor for the synthesis of cholesterol, as well as for the synthesis of a variety of non-sterol compounds involved in post-translational modification of proteins such as farnesyl pyrophosphate and geranyl pyrophosphate; and for dolichol, which is required for the synthesis of glycoproteins involved in cell-to-cell communication and cell structure. It has long been known that patients receiving steroid hormones of adrenocortical origin at pharmacologically appropriate doses show increased incidence of infectious disease. U.S. Pat. No. 5,527,789 discloses a method of combating cancer by administering to a patient DHEA and ubiquinone, where the cancer is one that is sensitive to DHEA.

DHEA is a 17-ketosteroid which is quantitatively one of the major adrenocortical steroid hormones found in mammals. Although DHEA appears to serve as an intermediary in gonadal steroid synthesis, the primary physiological function of DHEA has not been fully understood. It has been known, however, that levels of this hormone begin to decline in the second decade of life (reaching 5% of the original level in the elderly.) Clinically, DHEA has been used systemically and/or topically for treating patients suffering from psoriasis, gout, hyperlipemia, and it has been administered to post-coronary patients. In mammals, DHEA has been shown to have weight optimizing and anti-carcinogenic effects, and it has been used clinically in Europe in conjunction with estrogen as an agent to reverse menopausal symptoms and also has been used in the treatment of manic depression, schizophrenia, and Alzheimer's disease. DHEA has been used clinically at 40 mg/kg/day in the treatment of advanced cancer and multiple sclerosis. Mild androgenic effects, hirsutism, and increased libido were the side effects observed. These side effects can be overcome by monitoring the dose and/or by using analogues. The subcutaneous or oral administration of DHEA to improve the host's response to infections is known, as is the use of a patch to deliver DHEA. DHEA is also known as a precursor in a metabolic pathway which ultimately leads to more powerfuil agents that increase immune response in mammals. That is, DHEA acts as a prodrug: it acts as an immuno-modulator when converted to androstenediol or androst-5-ene-3β,17β-diol (βAED), or androstenetriol or androst-5-ene-3β,7β,17β-triol (βAET). However, in vitro DHEA has certain lymphotoxic and suppressive effects on cell proliferation prior to its conversion to βAED and/or βAET. It is, therefore, believed that the superior immunity enhancing properties obtained by administration of DHEA result from its conversion to more active metabolites.

Adenosine is a purine involved in intermediary metabolism, and may constitute an important mediator in the lung for various diseases, including bronchial asthma, COPD, CF, RDS, rhinitis, pulmonary fibrosis, and others. The potential role of its receptor was suggested by the finding that asthmatics respond to aerosolized adenosine with marked bronchoconstriction whereas normal individuals do not. An asthmatic rabbit animal model, the dust mite allergic rabbit model for human asthma, responded in a similar fashion to aerosolized adenosine with marked bronchoconstriction whereas non-asthmatic rabbits showed no response. More recent work with this animal model suggested that adenosine-induced bronchoconstriction and bronchial hyperresponsiveness in asthma may be mediated primarily through the stimulation of adenosine receptors. Adenosine has also been shown to cause adverse effects, including death, when administered therapeutically for other diseases and conditions in subjects with previously undiagnosed hyper-reactive airways. Adenosine plays a unique role in the body as a regulator of cellular metabolism. It can raise the cellular level of AMP, ADP and ATP that are the energy intermediates of the cell. Adenosine can stimulate or down regulate the activity of adenylate cyclase and hence regulate cAMP levels. cAMP, in turn, plays a role in neurotransmitter release, cellular division and hormone release. Adenosine's major role appears to be to act as a protective injury autocoid. In any condition in which ischemia, low oxygen tension or trauma occurs adenosine appears to play a role. Defects in synthesis, release, action and/or degradation of adenosine have been postulated to contribute to the over activity of the brain excitatory amino acid neurotransmitters, and hence various pathological states. Adenosine has also been implicated as a primary determinant underlying the symptoms of bronchial asthma and other respiratory diseases, the induction of bronchoconstriction and the contraction of airway smooth muscle. Moreover, adenosine causes bronchoconstriction in asthmatics but not in non-asthmatics. Other data suggest the possibility that adenosine receptors may also be involved in allergic and inflammatory responses by reducing the hyperactivity of the central dopaminergic system. It has been postulated that the modulation of signal transduction at the surface of inflammatory cells influences acute inflammation. Adenosine is said to inhibit the production of super-oxide by stimulated neutrophils. Recent evidence suggests that adenosine may also play a protective role in stroke, CNS trauma, epilepsy, ischemic heart disease, coronary by-pass, radiation exposure and inflammation. Overall, adenosine appears to regulate cellular metabolism through ATP, to act as a carrier for methionine, to decrease cellular oxygen demand and to protect cells from ischemic injury. Adenosine is a tissue hormone or inter-cellular messenger that is released when cells are subject to ischemia, hypoxia, cellular stress, and increased workload, and or when the demand for ATP exceeds its supply. Adenosine is a purine and its formation is directly linked to ATP catabolism. It appears to modulate an array of physiological processes including vascular tone, hormone action, neural function, platelet aggregation and lymphocyte differentiation. It also may play a role in DNA formation, ATP biosynthesis and general intermediary metabolism. It is suggested that it regulates the formation of cAMP in the brain and in a variety of peripheral tissues. Adenosine regulates cAMP formation through two receptors A₁ and A₂. Via A₁ receptors, adenosine reduces adenylate cyclase activity, while it stimulates adenylate cyclase at A₂ receptors. The adenosine A₁ receptors are more sensitive to adenosine than the A₂ receptors. The CNS effects of adenosine are generally believed to be A₁-receptor mediated, where as the peripheral effects such as hypotension, bradycardia, are said to be A₂ receptor mediated.

A handful of medicaments have been used for the treatment of respiratory diseases and conditions, although in general they all have limitations. Amongst them are glucocorticoid steroids, leukotriene inhibitors, anti-cholinergic agents, anti-histamines, oxygen therapy, theophyllines, and mucolytics. Glucocorticoid steroids are the ones with the most widespread use in spite of their well documented side effects. Most of the available drugs are nevertheless effective in a small number of cases, and not at all when it comes to the treatment of asthma. No treatments are currently available for many of the other respiratory diseases. Theophylline, an important drug in the treatment of asthma, is a known adenosine receptor antagonist which was reported to eliminate adenosine-mediated bronchoconstriction in asthmatic rabbits. A selective adenosine A1 receptor antagonist, 8-cyclopentyl-1,3-dipropylxanthine (DPCPX) was also reported to inhibit adenosine-mediated bronchoconstriction and bronchial hyperresponsiveness in allergic rabbits. The therapeutic and preventative applications of currently available adenosine A1 receptor-specific antagonists are, nevertheless, limited by their toxicity. Theophylline, for example, has been widely used in the treatment of asthma, but is associated with frequent, significant toxicity (gastrointestinal, cardiovascular, neurological and biological disturbances) resulting from its narrow therapeutic dose range. DPCPX is far too toxic to be useful clinically. The fact that, despite decades of extensive research, no specific adenosine receptor antagonist is available for clinical use attests to the general toxicity of these agents.

In some people, allergen exposure can cause a reaction known as the allergic response. This occurs when allergens are inhaled into the respiratory tract (nose, throat and lungs) and attach to the mucous membranes. These allergens are seen by the immune system as foreign invaders and an immune response is produced as the body prepares to fight them off. During this response, T-cells (a cell type of the immune system) send a signal to B-cells (B-lymphocytes) and stimulate production of IgE antibodies—a key protein involved in the allergic cascade. IgE antibodies, specific to the allergen, are produced within a few weeks after exposure and released into the bloodstream. These IgE antibodies may attach to receptors on inflammatory cells such as mast cells. Unattached IgE antibodies remain free floating in the bloodstreamn. When an allergic individual is re-exposed to an allergen, cross-linking to IgE bound on the mast cells may occur. When cross-linking occurs, mast cells release chemical mediators such as histamine, prostaglandins and leukotrienes. These chemical mediators can cause inflammatory responses in the body. These inflammatory responses have been linked to asthma signs and symptoms such as bronchial constriction, coughing and wheezing.

Genentech, Inc., Novartis Pharmaceuticals Corporation, an affiliate of Novartis AG, and Tanox, Inc. announced on Jun. 20, 2003 that the novel IgE-blocker Xolair® (Omalizumab) For Subcutaneous Use has been approved by the U.S. Food and Drug Administration (FDA). Xolair® is the first humanized therapeutic antibody for the treatment of asthma and the first approved therapy designed to target the antibody IgE. Xolair® is expected to be available by prescription beginning July 2003. Xolair® is the first humanized therapeutic antibody for the treatment of asthma and the first approved therapy designed to target the antibody IgE, a key underlying cause of the symptoms of allergy related asthma.

U.S. Pat. No. 5,660,835 (and corresponding PCT publication WO 96/25935) discloses a novel method of treating asthma or adenosine depletion in a subject by administering to the subject a dehydroepiandrosterone (DHEA) or DHEA-related compound. The patent also discloses a novel pharmaceutical composition in regards to an inhalable or respirable formulation comprising DHEA or DHEA-related compounds that is in a respirable particle size.

U.S. Pat. No. 5,527,789 discloses a method of combating cancer in a subject by administering to the subject a DHEA or DHEA-related compound, and ubiquinone to combat heart failure induced by the DHEA or DHEA-related compound.

U.S. Pat. No. 6,087,351 discloses an in vivo method of reducing or depleting adenosine in a subject's tissue by administering to the subject a DHEA or DHEA-related compound.

U.S. patent application Ser. No. 10/454,061, filed Jun. 3, 2003, discloses a method for treating COPD in a subject by administering to the subject a DHEA or DHEA-related compound.

U.S. patent application Ser. No. 10/462,901, filed Jun. 17, 2003, discloses a stable dry powder formulation of DHEA in a nebulizable form sealed in a container.

U.S. patent application Ser. No. 10/462,927, filed Jun. 17, 2003, discloses a stable dry powder formulation of dihydrate crystal form of DHEA-S suitable for treating asthma and COPD.

The above patents and patent applications are herein incorporated by reference in their entirety.

There exists a well defined need for novel and effective therapies for treating respiratory, lung and cancer ailments that cannot presently be treated, or at least for which no therapies are available that are effective and devoid of significant detrimental side effects. This is the case of ailments afflicting the respiratory tract, and more particularly the lung and the lung airways, including respiratory difficulties, asthma, bronchoconstriction, lung inflammation and allergies, depletion or hyposecretion of surfactant, etc. Moreover, there is a definite need for treatments that have prophylctic and therapeutic applications, and require low amounts of active agents, which makes them both less costly and less prone to detrimental side effects.

Further, there is a need to better ensure patient compliance in the taking of medication, and a need to facilitate the taking of the plurality of compounds necessary for prevention or treatment of asthma, COPD, or other respiratory diseases.

### SUMMARY OF THE INVENTION

The present invention provides for a composition comprising at least two active agents. A first active agent comprises a non-glucocorticoid steroid, such as an epiandrosterone (EA) or a salt thereof. A second active agent comprises an anti-IgE antibody. The composition comprises a combination of the first active agent and the second active agent. The amount of the first active agent and the amount of the second active agent in the composition is of an amount sufficient to effectively prophylactically or therapeutically treat a subject in danger of suffering or suffering from asthma, COPD, or other respiratory diseases when the composition is administered to the subject. The anti-IgE antibody is one that is capable of binding IgE in order to effectively prophylactically or therapeutically treat a subject in danger of suffering or suffering from asthma, COPD, or other respiratory diseases when the anti-IgE antibody is administered to the subject. The composition can further comprise other bioactive agents and formulation ingredients. The composition is a pharmaceutical or veterinary composition suitable for administration to a subject or patient, such as a human or a non-human animal (such as a non-human mammal).

The composition is useful for treating asthma, COPD, or other respiratory diseases for which inflammation and its sequelae plays a role including conditions associated with bronchoconstriction, surfactant depletion and/or allergies.

The present invention also provides for methods for treating asthma, COPD, or other respiratory diseases comprising administering the composition to a subject in need of such treatment.

The present invention also provides for a use of the first active agent and the second active agent in the manufacture of a medicament for the prophylactic or therapeutic treatment of asthma, COPD, or other respiratory diseases described above.

The present invention also provides for a kit comprising the composition and a delivery device. The delivery device is capable of delivering the composition to the subject. Preferably, the delivery device comprises an inhaler provided with an aerosol or spray generating means that delivers particles. Preferably, the delivery is to the airway of the subject. More preferably, the delivery is to the lung or lungs of the subject. Preferably, the delivery is directly to the desired location.

The main advantage of using the compositions is the compliance by the patients in need of such prophylaxis or treatment. Respiratory diseases such as asthma or COPD are multifactorial with different manifestations of signs and symptoms for individual patients. As such, most patients are treated with multiple medications to alleviate different aspects of the disease. A fixed combination of the first active agent, such as DHEA or DHEA-S, and the second active agent, such as E-25 (omalizumab), permits more convenient yet targeted therapy for a defined patient subpopulation. Patient compliances should be improved by simplifying therapy and by focusing on each patient's unique disease attributes so that their specific symptoms are addressed in the most expeditious fashion. Further, there is the added advantage of convenience or savings in time in the administering of both the first and second active agents in one administration. This is especially true when the composition is administered to a region of the body of the subject that has the potential of discomfort, such as the composition administered to the airways of the subject. This is also especially true when the administration of the compositions to the subject is invasive.

In addition, the first active agent, such as DHEA or DHEA-S, is an anti-inflammatory agent that is most effective when it is delivered or deposited in the distal peripheral airways rather than the conducting airways, in the alveolar membranes and fine airways. Asthma and some COPD patients have conducting airways that are constricted, which limit the delivery (due to earlier deposition caused by lower particle velocity) of the first active agent, such as DHEA, acting on these distal peripheral airways. Therefore, the combination of a bronchodilator drug (β2 agonist, antimuscarinic which reverses elevated tone) facilitates the delivery of an anti-inflammatory to the distal peripheral airways. Use of the combination provides an improved sustained pharmacologic effect that translates an improved disease management. The antileukotrienes reduce interstitial edema in the very small peripheral airways. This too would have the effect of increasing peripheral airway diameter and facilitate delivery of the first active agent. This is also true for antihistamines, which also reduce peripheral airways edema and facilitate distal airway delivery of the first active agent.

The drawings accompanying this patent form part of the disclosure of the invention, and further illustrate some aspects of the present invention as discussed below.

### BRIEF DESCRIPTION OF THE DRAWINGS

FIG. 1 depicts fine particle fraction of neat micronized DHEA-S.2H₂O delivered from the single-dose Acu-Breathe inhaler as a function of flow rate. Results are expressed as DHEA-S. IDL data on virtually anhydrous micronized DHEA-S are also shown in this figure where the 30 L/min result was set to zero since no detectable mass entered the impactor.

FIG. 2 depicts HPLC chromatograms of virtually anhydrous DHEA-S bulk after storage as neat and lactose blend for 1 week at 50° C. The control was neat DHEA-S stored at room temperature (RT)

FIG. 3 depicts HPLC chromatograms for DHEA-S.2H₂O bulk after storage as neat and lactose blend for 1 week at 50° C. The control was neat DHEA-S.2H₂O stored at RT.

FIG. 4 depicts solubility of DHEA-S as a function of NaCl concentration at two temperatures.

FIG. 5 depicts DHEA-S solubility as a function of the reciprocal sodium cation concentration at 24-25° C.

FIG. 6 depicts DHEA-S solubility as a function of the reciprocal sodium cation concentration at 7-8° C.

FIG. 7 depicts solubility of DHEA-S as a function of NaCl concentration with and without buffer at RT.

FIG. 8 depicts DHEA-S solubility as a function of the reciprocal of sodium cation concentration at 24-25° C. with and without buffer.

FIG. 9 depicts solution concentration of DHEA-S versus time at two storage conditions.

FIG. 10 depicts solution concentration of DHEA versus time at two storage conditions.

FIG. 11 depicts the schematic for nebulization experiments.

FIG. 12 depicts mass of DHEA-S deposited in by-pass collector as a function of initial solution concentration placed in the nebulizer.

FIG. 13 depicts particle size by cascade impaction for DHEA-S nebulizer solutions. The data presented are the average of all 7 nebulization experiments.

FIG. 14 depicts the inhibition of HT-29 SF cells by DHEA.

FIG. 15 depicts the effects of DHEA on cell cycle distribution in HT-29 SF cells.

FIGS. 16a and 16b depict the reversal of DHEA-induced growth inhibition in HT-29 cells.

FIG. 17 depicts the reversal of DHEA-induced G₁ arrest in HT-29 SF cells.

FIG. 18 depicts a sequence comparison of the differences between the light chain and heavy chain CDR domains between rhuMabe25, e426, and sequences e26 and e27. The residue numbering here is consecutive, as opposed to that of Kabat et al. These sequences are only fragments and do not show the full-length heavy and light chain residues. The variable light chain sequence of e26 and e27 is represented by SEQ ID NO: 1. The variable light chain sequence of e426 is represented by SEQ ID NO:2. The variable light chain sequence of e25 is represented by SEQ ID NO:3. The variably heavy chain sequence of e27 is represented by SEQ ID NO:4. The variable heavy chain sequence of e25, e26 and e426 is represented by SEQ ID NO:5.

FIG. 19 depicts the full length heavy and light chain residues of anti-IgE antibodies E25, E26 and E27. The full length variable light chain sequence of e25 is represented by SEQ ID NO:6. The full length heavy chain sequence of e25 is represented by SEQ ID NO:7. The full length light chain sequence of e26 is represented by SEQ ID NO:8. The full length heavy chain sequence of e26 is represented by SEQ ID NO:9. The full length light chain sequence of e27 is represented by SEQ ID NO: 10. The full length heavy chain sequence of e27 is represented by SEQ If) NO: 11.

FIG. 20 depicts the F(ab) fragments of anti-IgE antibodies e26 and e27. The light chain sequence of a e26 and Fab fragment of e27 is represented by SEQ ID NO: 12. The heavy chain sequence of a Fab fragment of e26 is represented by SEQ ID NO: 13. The heavy chain sequence of a Fab fragment of e27 is represented by SEQ ID NO:14.

FIG. 21 depicts the sFV fragments of anti-IgE antibodies e26 and e27. The sFv fragment of e26 is represented by SEQ ID NO: 15. The sFv fragment of e27 is represented by SEQ ID NO:16.

FIG. 22 depicts the F(ab′)₂ fragments of anti-IgE antibodies e26 and e27. The light chain sequence of a F(ab′)₂ fragment of e26 and e27 is represented by SEQ ID NO: 17. The heavy chain sequence of a F(ab′)₂ fragment of e26 is represented by SEQ ID NO: 18. The heavy chain sequence of a F(ab′)₂ fragment of e27 is represented by SEQ ID NO: 19.

FIG. 23 depicts certain suitable analogs of DHEA.

FIG. 24 depicts certain suitable analogs of DHEA.

FIG. 25 depicts certain suitable analogs of DHEA.

FIG. 26 depicts suitable modifications of the C-17 ketone of DHEA.

### DETAILED DESCRIPTION OF THE PREFERRED EMBODIMENTS

Definitions

In the present context, the terms “adenosine” and “surfactant” depletion are intended to encompass levels that are lowered or depleted in the subject as compared to previous levels in that subject, and levels that are essentially the same as previous levels in that subject but, because of some other reason, a therapeutic benefit would be achieved in the patient by modification of the levels of these agents as compared to previous levels.

The term “airway”, as used herein, means part of or the whole respiratory system of a subject that is exposed to air. The airway includes, but not exclusively, throat, tracheobronchial tree, nasal passages, sinuses, among others. The airway also includes trachea, bronchi, bronchioles, terminal bronchioles, respiratory bronchioles, alveolar ducts, and alveolar sacs.

The term “airway inflammation”, as used herein, means a disease or condition related to inflammation on airway of subject. The airway inflammation may be caused or accompanied by allergy(ies), asthma, impeded respiration, cystic fibrosis (CF), Chronic Obstructive Pulmonary Diseases (COPD), allergic rhinitis (AR), Acute Respiratory Distress Syndrome (ARDS), microbial or viral infections, pulmonary hypertension, lung inflammation, bronchitis, cancer, airway obstruction, and bronchoconstriction.

The term “carrier”, as used herein, means a biologically acceptable carrier in the form of a gaseous, liquid, solid carriers, and mixtures thereof, which are suitable for the different routes of administration intended. Preferably, the carrier is pharmaceutically or veterinarily acceptable.

“An effective amount” as used herein, means an amount which provides a therapeutic or prophylactic benefit.

“Other therapeutic agents” refers to any therapeutic agent is not the first or second active agent of the composition.

The terms “prophylaxis”, as used herein, mean a prophylactic treatment made before a subject experiences a disease or a worsening of a previously diagnosed condition such that it can have a subject avoid, prevent or reduce the probability of having a disease symptom or condition related thereto. The subject can be one of increased risk of obtaining the disease or a worsening of a previously diagnosed condition.

The term “respiratory diseases”, as used herein, means diseases or conditions related to the respiratory system. Examples include, but not limited to, airway inflammation, allergy(ies), impeded respiration, cystic fibrosis (CF), allergic rhinitis (AR), Acute Respiratory Distress Syndrome (ARDS), cancer, pulmonary hypertension, lung inflammation, bronchitis, airway obstruction, bronchoconstriction, microbial infection, and viral infection, such as SARS.

The terms “treat”, “treating” or “therapeutic”, as used herein, mean a treatment which decreases the likelihood that the subject administered such treatment will manifest symptoms of disease or other conditions.

The present invention provides for a composition comprising a first active agent comprising a non-glucocorticoid steroid, such as an epiandrosterone (EA), analogue thereof, or a salt thereof (preferably DHEA or DHEA-S), in combination with a second active agent comprising an anti-IgE antibody. The composition can further comprise a pharmaceutical or veterinarily acceptable carrier, diluent, excipient, bioactive agent or ingredient. The compositions are useful for treating asthma, COPD, or other respiratory diseases. Other respiratory diseases that the compositions are also useful for treating are lung and respiratory diseases and conditions associated with bronchoconstriction, lung inflammation and/or allergies, and lung cancer.

The first active agent is an epiandrosterone, an analogue or a pharmaceutically or veterinarily acceptable salt thereof. The epiandrosterone, an analogue or a pharmaceutically or veterinarily acceptable salt thereof is selected from a non-glucocorticoid steroid having the chemical formula 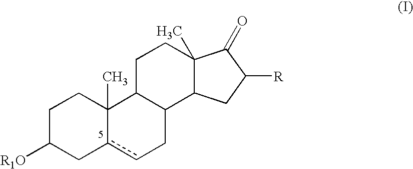
wherein the broken line represents a single or a double bond; R is hydrogen or a halogen; the H at position 5 is present in the alpha or beta configuration or the compound of chemical formula I comprises a racemic mixture of both configurations; and R¹ is hydrogen or a multivalent inorganic or organic dicarboxylic acid covalently bound to the compound; a non-glucocorticoid steroid of the chemical formula 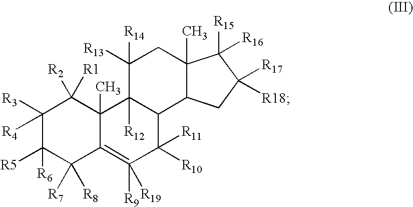
a non-glucocorticoid steroid of the chemical formula 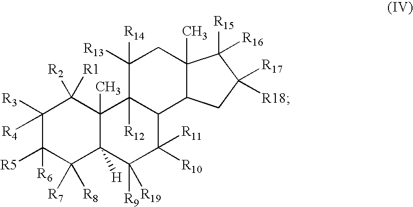
or a combination thereof wherein R1, R2, R3, R4, R5, R7, R8, R9, R10, R12, R13, R14 and R19 are independently H, OR, halogen, (C1-C10) alkyl or (C1-C10) alkoxy, R5 and R11 are independently OH, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO2R20, —OPOR2OR21 or (C1-C10) alky, R5 and R6 taken together are ═O, R10 and R11 taken together are ═O; R15 is (1) H, halogen, (C1-C10) alkyl, or (C1-C10) alkoxy when R16 is —C(O)OR22, (2) H, halogen, OH or (C1-C10) alkyl when R16 is halogen, OH or (C1-C10) alkyl, (3) H, halogen, (C1-C10) alkyl, (C1-C10) alkenyl, (C1-C10) alkynyl, formyl, (C1-C10) alkanoyl or epoxy when R16 is OH, (4) OR, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO2R20 or —OPOR2OR21 when R16 is H, or R15 and R16 taken together are ═O; R17 and R18 are independently (1) H, —OH, halogen, (C1-C10) alkyl or —(C1-C10) alkoxy when R6 is H OR, halogen. (C1-C10) alkyl or —C(O)OR22, (2) H, (C1-C10 alkyl).amino, ((C1-C10) alkyl)ₙ amino-(C1-C10) alkyl, (C1-C10) alkoxy, hydroxy-(C1-C10) alkyl, (C1-C10) alkoxy-(C1-C10) alkyl, (halogen)m (C1-C10) alkyl, (C1-C10) alkanoyl, formyl, (C1-C10) carbalkoxy or (C1-C10) alkanoyloxy when R15 and R16 taken together are ═O, (3) R17 and R18 taken together are ═O; (4) R17 or R18 taken together with the carbon to which they are attached form a 3-6 member-ring containing 0 or 1 oxygen atom; or (5) R15 and R17 taken together with the carbons to which they are attached form an epoxide ring; R20 and R21 are independently OH, pharmaceutically acceptable ester or pharmaceutically acceptable ether; R22 is H, (halogen)ₘ (C1-C10) alkyl or (C1-C10) alkyl; n is 0, 1 or 2; and m is 1, 2 or 3; or pharmaceutically or veterinarily acceptable salts thereof.

Preferably, for chemical formula (I), the multivalent organic dicarboxylic acid is SO₂OM, phosphate or carbonate, wherein M comprises a counterion. Examples of a counterion are H, sodium, potassium, magnesium, aluminum, zinc, calcium, lithium, ammonium, amine, arginine, lysine, histidine, triethylamine, ethanolamine, choline, triethanoamine, procaine, benzathine, tromethanine, pyrrolidine, piperazine, diethylamine, sulfatide 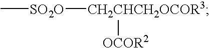
and phosphatide 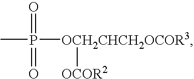
wherein R² and R³, which may be the same or different, are straight or branched (C₁-C₁₄) alkyl or glucuronide

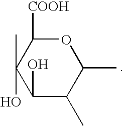

The hydrogen atom at position 5 of the chemical formula I may be present in the alpha or beta configuration, or the DHEA compound may be provided as a mixture of compounds of both configurations. Compounds illustrative of chemical formula I above are included, although not exclusively, are DHEA, wherein R and R¹ are each hydrogen, containing a double bond, 16-alpha bromoepiandrosterone, wherein R is Br, R¹ is H, containing a double bond; 16-alpha-fluoro epiandrosterone, wherein R is F, R¹ is H, containing a double bond; Etiocholanolone, wherein R and R¹ are each hydrogen lacking a double bond; and dehydroepiandrosterone sulphate, wherein R is H, R¹ is SO₂OM and M is a sulphatide group as defined above, lacking a double bond. Others, however, are also included. Also preferred compounds of formula I are those where R is halogen, e.g. bromo, chloro, or fluoro, where R1 is hydrogen, and where the double bond is present. A most preferred compound of formula I is 16-alpha-fluoro epiandrosterone. Other preferred compounds are DHEA and DHEA salts, such as the sulfate salt (DHEA-S).

In general, the non-glucocorticoid steroid, such as those of formulas (I), (III) and (IV), their derivatives and their salts are administered in a dosage of about 0.05, about 0.1, about 1, about 5, about 20 to about 100, about 500, about 1000, about 1500 about 1800, about 2500, about 3000, about 3600 mg/kg body weight. Other dosages, however, are also suitable and are contemplated within this patent. The first active agent of formula (I), (1E) and (IV) may be made in accordance with known procedures, or variations thereof that will be apparent to those skilled in the art. See, for example, U.S. Pat. No. 4,956,355; UK Patent No. 2,240,472; EPO Patent Application No. 429; 187, PCT Patent Publication No. WO 91/04030; U.S. Pat. No. 5,859,000; Abou-Gharbia et al., J. Pharm. Sci. 70: 1154-1157 (1981); Merck Index Monograph No. 7710 (11th Ed. 1989), among others.

In some embodiments of the invention, the first active agent can be an epiandrosterone analog or derivative thereof. Also, prodrugs and active metabolites of epiandrosterone are encompassed by the present invention. Those of skill in the art will recognize that the compounds described herein may exhibit the phenomena of tautomerism, conformational isomerism, geometric isomerism and/or optical isomerism. It should be understood that the invention encompasses any tautomeric, conformational isomeric, optical isomeric, and/or geometric isomeric forms of the compounds having one or more of the utilities described herein, as well as mixtures of these various different forms.

Metabolites of epiandrosterones, such as those described in the following references maybe used as the first active agent—Capillary gas chromatography of urinary steroids of terbutaline-treated asthmatic children, Chromatographia (1998), 48(1/2), 163-165; Androstenedione metabolism in human lung fibroblasts; Journal of Steroid Biochemistry (1986), 24(4), 893-7; Metabolism of androsterone and 5α-androstane-3α,17β-diol in human lung tissue and in pulmonary endothelial cells in culture, Journal of Clinical Endocrinology and Metabolism (1985), 60(2), 244-50; Testosterone metabolism by human lung tissue, Journal of Steroid Biochemistry (1978), 9(1), 29-32; Metabolism of androsterone and 5 alpha-androstane-3 alpha,17 beta-diol in human lung tissue and in pulmonary endothelial cells in culture, Journal of clinical endocrinology and metabolism (1985 February), 60(2), 44-50; Metabolism of dehydroisoandrosterone and androstenedione in human pulmonary endothelial cells in culture, Journal of clinical endocrinology and metabolism (1983 May), 56(5), 930-5; Metabolism of dehydroisoandrosterone and androstenedione by the human lung in vitro, Journal of steroid biochemistry (1977 April), 8(4), 277-84; and Testosterone metabolism in dog lung in vitro, Steroids and lipids research (1973), 4(1), 17-23, which references are herein incorporated by reference in their entirety.

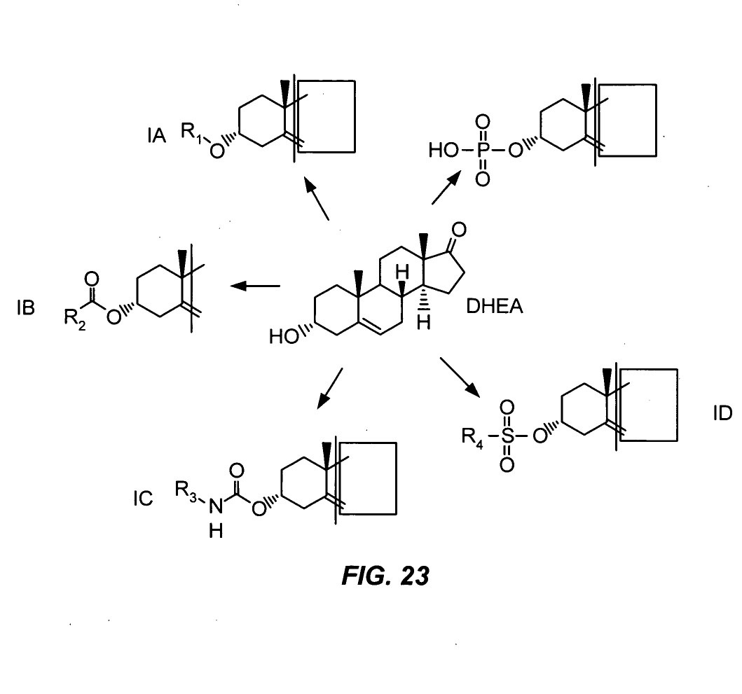

Other suitable analogs of DHEA that can be used as the first active agent are described herein. FIG. 23 depicts certain suitable analogs of DHEA, including compounds of the Formulas IA, IB, IC, and ID. In the depiction of suitable R groups herein, the attachment point is indicated by a CH2 group or by an atom marked with an asterisk. R1 and R3 can be linear or branched alkyls including benzyl and optionally substituted alkyls, such as aminoalkyls, hydroxyalkyls, ethers, and carboxylic acids, and optionally substituted aryl and heteroaryls. R1 and R3 can be, for example,
CF₃ CH₃(CH₂)ₙ where n is preferably 0 to 4 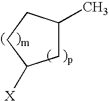
   where X=OH, O-alkyl, N (substituted by H, alkyl, or ac) and m and p are independently 1-4 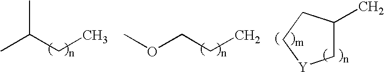
   Where Y=O, NH, N-alkyl or N-acyl 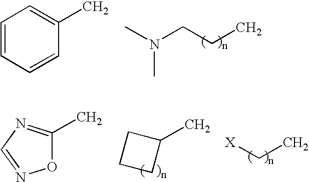
   where X=CO₂H or amide
      Examples of compounds of formula IA include, 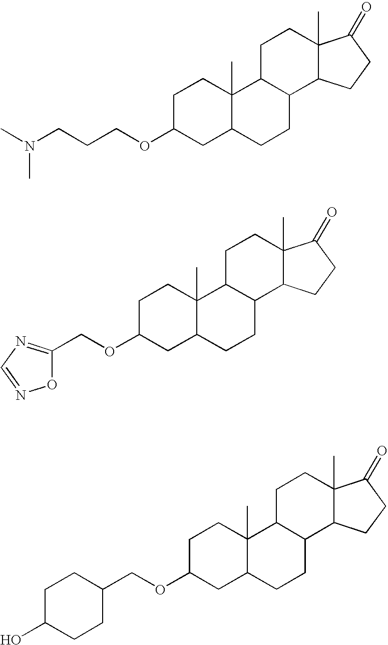
      R2 is preferably a diacid-derived or amino acid derived substituent, potentially including chloracetyl derivatives and acrylate derivatives, or optionally substituted aryls such as benzyl and heterobenzoyl. Examples of compounds of formula IB include, 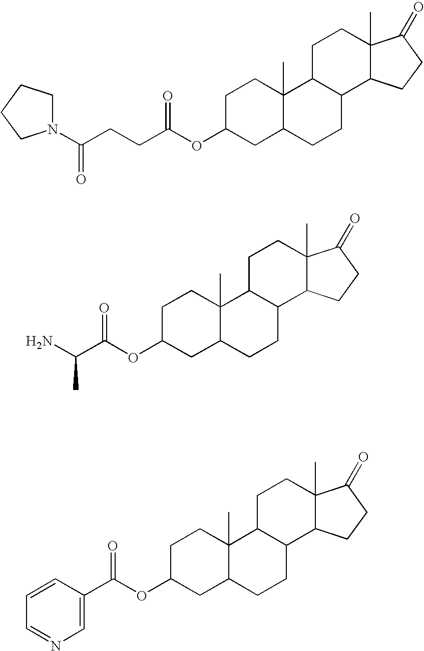
      Examples of compounds of formula IC include, 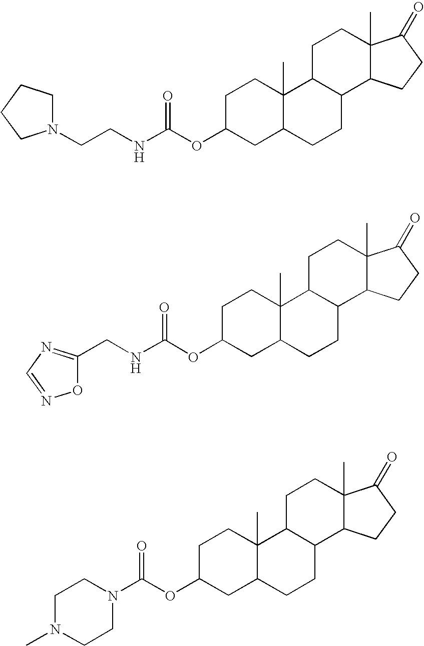
      R4 can be aromatic in nature and examples of suitable compounds of formula ID include,

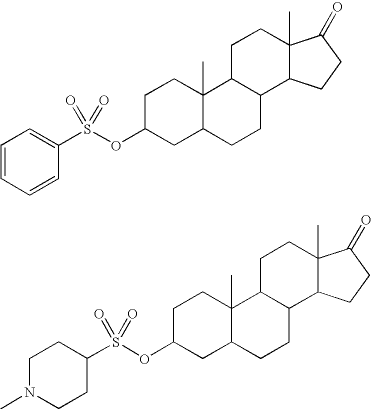

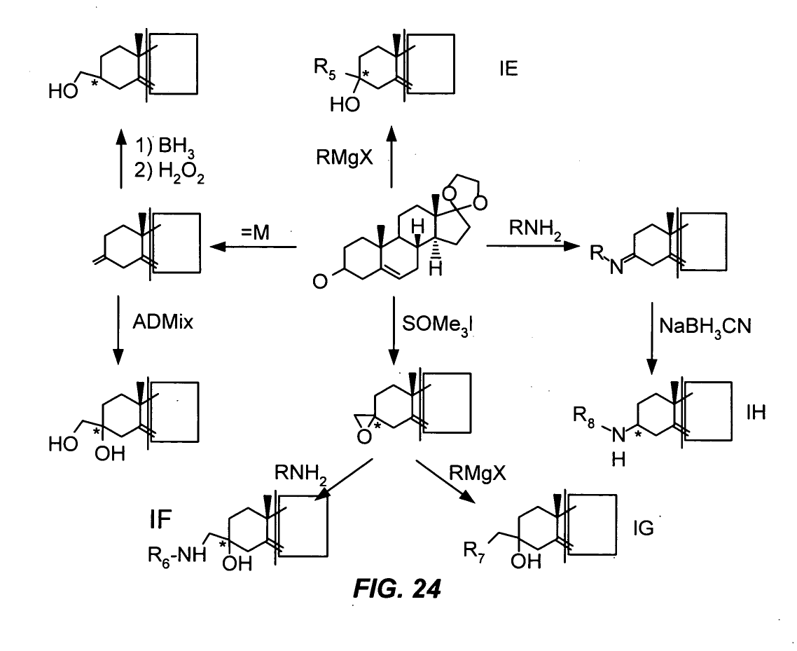

Other suitable analogs include the analogs wherein modifications have been performed at C-3 position by retaining OH or replacing OH with NH. These analogs are typically made by starting from C-17 acetal protected andrst-4-ene3, 17-dione, such as depicted in FIG. 24. Compounds of Formula IE can be derived from Grignard reagents and possibly aryl-lithium reagents, such as aromatic in nature, and can also be alkynyl, alkenyl, and alkyl. Examples of R5 are
CH₃(CH₂)ₙ where n=0 to 4 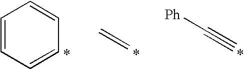
   Examples of compounds of Formula IE include 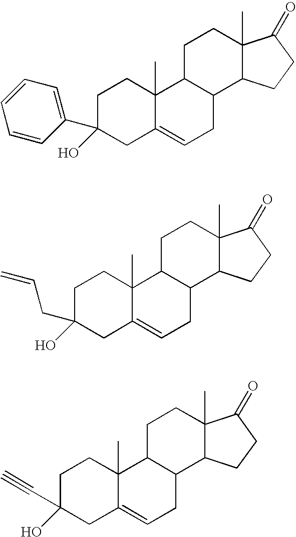
   R6 and R8 can independently be a diverse set of amines and can include amines possessing the functionalities as described for the R1 group. Examples of suitable compounds of Formula IF include, 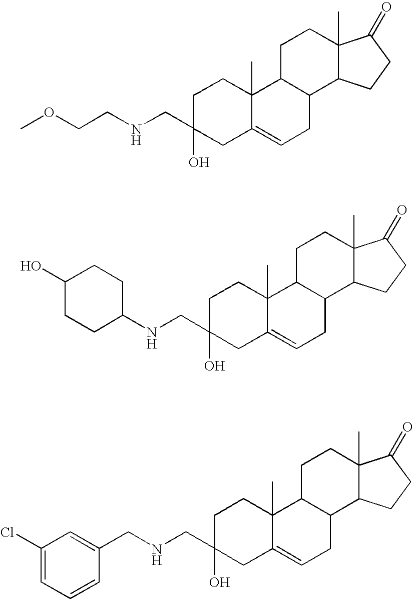
   Suitable R7 groups can be derived from Grignard/organolithium reagents and so could encompass the functionalities described for R5. Examples of compounds of Formula IG include 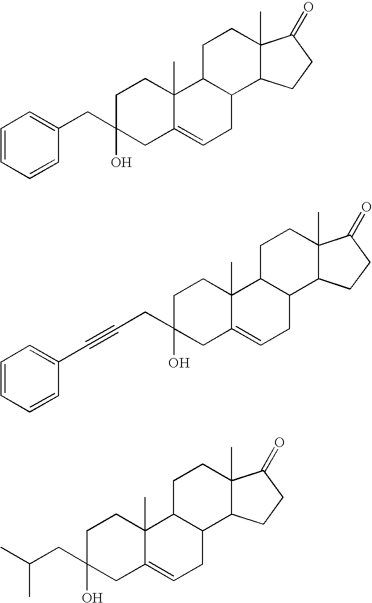
   Examples of compounds of Formula IH include

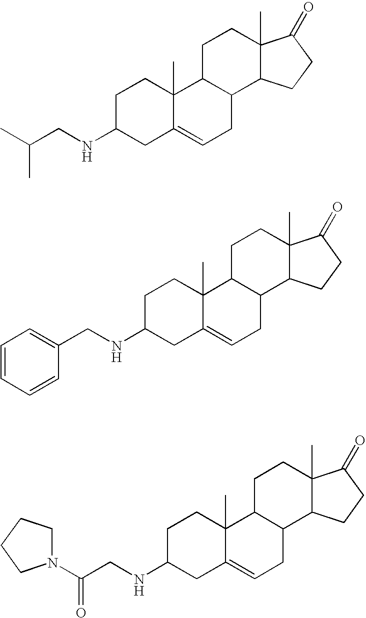

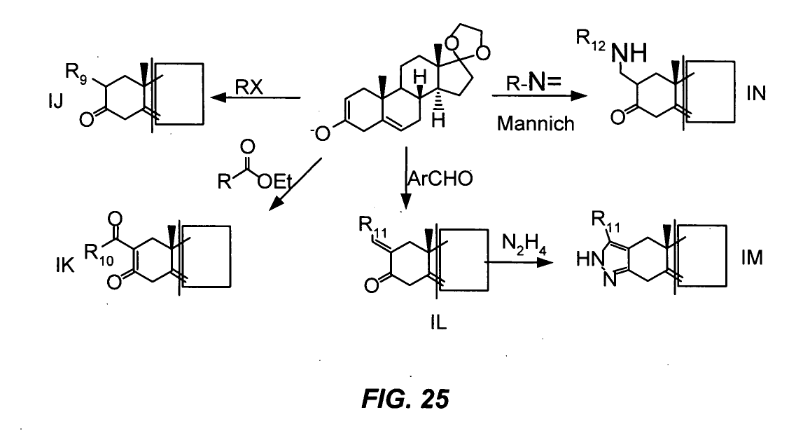

Other suitable analogs include those compounds wherein the C-2 position of DHEA was modified. Suitable modifications are depicted in FIG. 25. R9 can be derived from alkylating agents, such as alkyl, benzyl, heterobenzyl, and derivatives of other activated halides. Examples of R9 include, 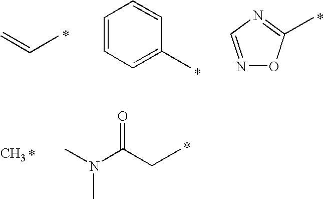
Example of Formula U compounds include, 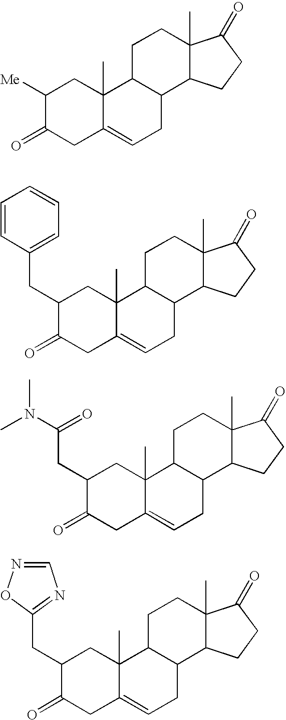
R10 can be aromatic esters such as with aryl or heteroaryl ring or enolisable alkyl esters. Examples of compounds of formula IK include, 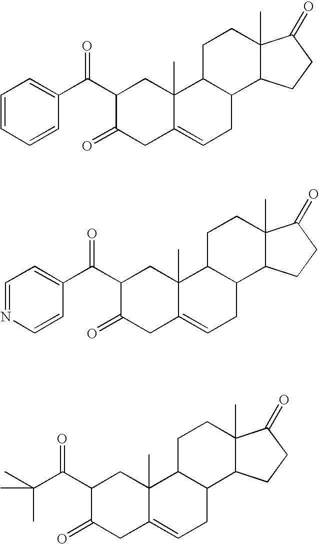
R11 can be a series of aromatic and heteroaromatic aldehydes such as benzene carboxaldehyde and substituted variants thereof, pyridine carboxaldehyde or non-enolisable aldehydes such as (CH3)3CCH═O. Examples of compounds of Formula IL include, 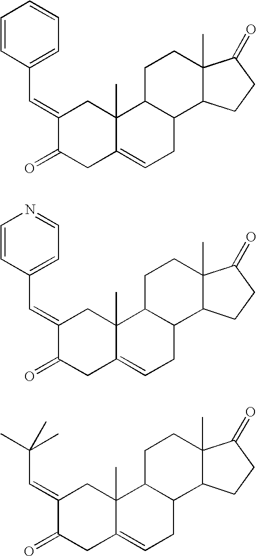
R12 can be a subset of an amine such as for R6. Examples of compounds of formula IN include,

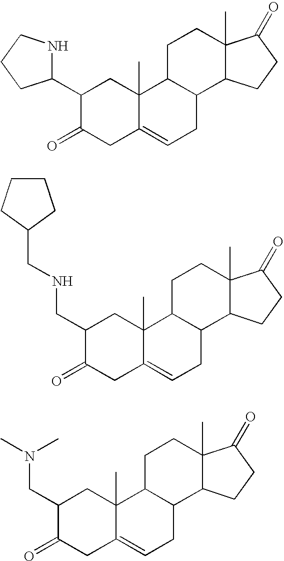

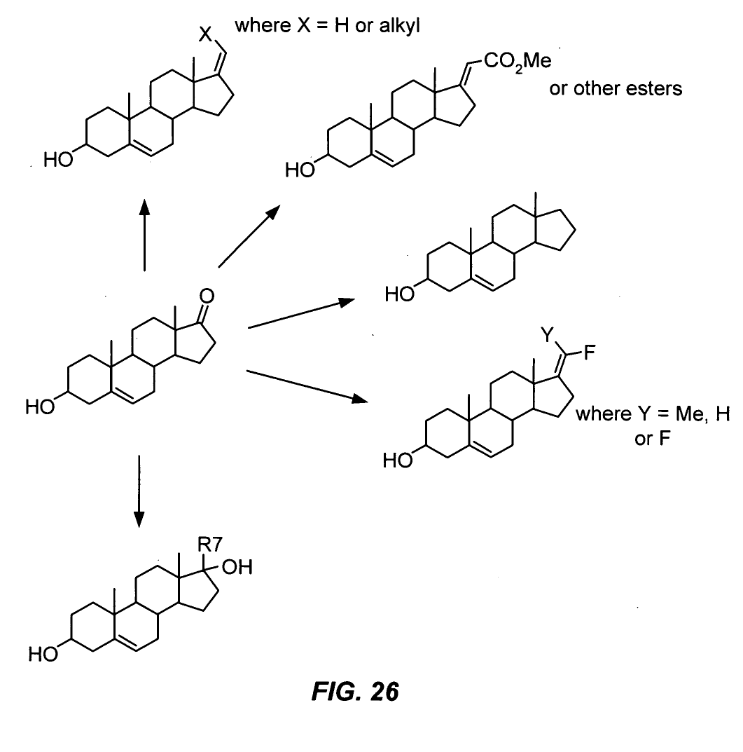

Suitable modifications of the C-17 ketone of DHEA are depicted in FIG. 26. The compounds depicted in FIG. 23 can also be used as the first active agent.

Other suitable DHEA analogs are described in U.S. Pat. No. 6,635,629; European Patent 934745; Dehydroepiandrosterone and analogs inhibit DNA binding of AP-1 and airway smooth muscle proliferation, Journal of Pharmacology and Experimental Therapeutics (1998), 285(2), 876-883; and Dehydroepiandrosterone and related steroids inhibit mitochondrial respiration in vitro, International Journal of Biochemistry (1989), 21(10), 1103-7, all of which are herein incorporated by reference in their entirety.

The second active agent is an anti-IgE antibody, or any functional fragment or antibody fragment thereof, capable of binding to IgE and preventing the IgE mediated allergic cascade. The anti-IgE antibody is capable of inhibiting any IgE-mediated disorder. Preferably, the anti-IgE antibody inhibits the IgE-mediated production of histamine. The range of anti-IgE antibody encompassed by this invention encompasses the compounds defined in U.S. Pat. Nos. 5,543,144; 5,422,258; 5,653,980; 5,945,294; 5,965,709; and, 5,994,511 (the disclosure of which are incorporated herein by reference). Examples of the anti-IgE antibody are the antibody E-25 (or omalizumab), E-26, and E-27.

Preferably, the anti-IgE antibody is the anti-IgE antibody E-25 or omalizumab. More preferably, the anti-IgE antibody E-25 is modified by replacement of VL CDR-L1 residue 32Asp with Glu, along with the modification of VL CDR-L1 residues 27Gln, 28 Ser and 31 Tyr to Lys, Pro and Gly, respectively. Even more preferrably, the E-25 anti-IgE antibody has additional modifications at residues VH CDR2: 53Thr to Lys, 55Asp to Ser, 57Ser to Glu and 59Asn to Lys. Preferably, the anti-IgE antibody has an improved affinity to IgE. Preferably, the anti-IgE antibody comprises heavy and light chain residues comprising the sequence fragments labeled “e27” and “e26”. Alternatively, the anti-IgE antibody comprises the full length heavy and light chain sequences labeled “E27” and “E26”. Preferably, the anti-IgE antibody is capable of reducing or inhibiting the IgE-mediated production of histamine.

In one embodiment, the anti-IgE antibody comprises, or consists, the sequence “e25” of SEQ ID NOs:6-7. In another embodiment, the anti-IgE antibody comprises, or consists, the sequence “e26” of SEQ ID NOs:8-9. In another embodiment, the anti-IgE antibody comprises, or consists, the sequence “e27” of SEQ ID NOs:10-11. In another embodiment, the anti-IgE antibody comprises an e26 sequence selected from the group consisting of: Fab fragment (SEQ ID NOs:12-13), sFv fragment (SEQ ID NO:15) and F(ab′)₂ (SEQ ID NOs:17-18). In another embodiment, the anti-IgE antibody comprises an e27 sequence selected from the group consisting of Fab fragment (SEQ ID NOs:12-13), sFv fragment (SEQ ID NO:16) and F(ab′)₂ (SEQ ID NOs:17 and 19).

The anti-IgE antibodies of this present invention can be prepared using the methods disclosed in U.S. Pat. Nos. 5,543,144; 5,422,258; 5,653,980; 5,945,294; 5,965,709; and, 5,994,511 (the disclosure of which are incorporated herein by reference).

The terms “protein” or “polypeptide” are intended to be used interchangeably. They refer to a chain of two (2) or more amino acids which are linked together with peptide or amide bonds, regardless of post-translational modification (e.g., glycosylation or phosphorylation). Antibodies are specifically intended to be within the scope of this definition. The polypeptides of this invention may comprise more than one subunit, where each subunit is encoded by a separate DNA sequence.

The phrase “substantially identical” with respect to an antibody polypeptide sequence shall be construed as an antibody exhibiting at least 70%, preferably 80%, more preferably 90% and most preferably 95% sequence identity to the reference polypeptide sequence. The term with respect to a nucleic acid sequence shall be construed as a sequence of nucleotides exhibiting at least about 85%, preferably 90% more preferably 95% and most preferably 97% sequence identity to the reference nucleic acid sequence. For polypeptides, the length of the comparison sequences will generally be at least 25 amino acids. For nucleic acids, the length will generally be at least 75 nucleotides.

The term “identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)₂ and Fv) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VI H) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny et al., Proc. Natl. Acad. Sci. USA 82, 4592-4596 (1985) (the disclosure of which are incorporated herein by reference).

An “isolated” antibody is one which has been identified and separated and/or recovered from a component of the environment in which is was produced. Contaminant components of its production environment are materials which would interfere with diagnostic or therapeutic uses for the antibody, and may include enzymes, hormones, and other proteinaceous or nonproteinaceous solutes. In preferred embodiments, the antibody will be purified as measurable by at least three different methods: 1) to greater than 95% by weight of antibody as determined by the Lowry method, and most preferably more than 99% by weight; 2) to a degree sufficient to obtain at least 15 residues of N-terminal or internal amino acid sequence by use of a spinning cup sequentator; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1×10⁻⁷ M, preferably no more than about 1.times.10.sup.-8 and most preferably no more than about 1×10⁻⁹ M) but has a binding affinity for a homologue of the antigen from a second non-human mammalian species which is at least about 50 fold, or at least about 500 fold, or at least about 1000 fold, weaker than its binding affinity for the human antigen. The species-dependent antibody can be of any of the various types of antibodies as defined above, but preferably is a humanized or human antibody.

The term “antibody mutant” refers to an amino acid sequence variant of an antibody wherein one or more of the amino acid residues have been modified. Such mutant necessarily have less than 100% sequence identity or similarity with the amino acid sequence having at least 75% amino acid sequence identity or similarity with the amino acid sequence of either the heavy or light chain variable domain of the antibody, more preferably at least 80%, more preferably at least 85%, more preferably at least 90%, and most preferably at least 95%. Since the method of the invention applies equally to both polypeptides, antibodies and fragments thereof, these terms are sometimes employed interchangeably.

The term “variable” in the context of variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877). With respect to Applicants' anti-IgE antibody, certain CDRs were defined by combining the Kabat et al. and Chothia et al. approaches. The more highly conserved portions of variable domains are called the framework (FR). The variable domains of native heavy and light chains each comprise four FR regions, largely a adopting a .beta.-sheet configuration, connected by three CDRs, which form loops connecting, and in some cases forming part of, the .beta.-sheet structure. The CDRs in each chain are held together in close proximity by the FR regions and, with the CDRs from the other chain, contribute to the formation of the antigen binding site of antibodies (see Kabat et al.) The constant domains are not involved directly in binding an antibody to an antigen, but exhibit various effector function, such as participation of the antibody in antibody-dependent cellular toxicity.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

An “Fv” fragment is the minimum antibody fragment which contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment (also designated as F(ab)) also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

The light chains of antibodies (immunoglobulin) from any vertebrate species can be assigned to one of two clearly distinct types, called kappa (K) and lambda (X), based on the ainino sequences of their constant domain.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson et al. Nature 352: 624-628 (1991), as well as in Marks et al., J. Mol. Biol. 222: 581-597 (1991). (The disclosure of these references and patent are incorporated herein by reference.)

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al. Proc. Natl. Acad. Sci. 81, 6851-6855 (1984).

“Humanized” forms of non-human (e.g. murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and corresponding non-human residues. Furthermore, humanized antibody may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunolglobulin. For further details, see: Jones et al., Nature 321, 522-525 (1986); Reichmann et al., Nature 332, 323-329 (1988) and Presta, Curr. Op. Struct. Biol. 2, 593-596 (1992) (the disclosure of which are incorporated herein by reference.).

“Single-chain Fv” or “sFv” antibody fragments comprise the VH and VL domains of an antibody, wherein these domains are present in a single polypeptide chain. Generally, the Fv polypeptide further comprises a polypeptide linker between the VH and VL domains which enables the sFv to form the desired structure for antigen binding. For a review of sFv see Pluckthun in The Pharmacology of Monoclonal Antibodies, vol. 113, Rosenburg et al. eds. Springer-Verlag, New York, pp. 269-315 (1994).

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad. Sci. USA 90: 6444-6448 (1993) (the disclosure of these references and patents are incorporated herein by reference.).

The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI.

The term “amino acid” and “amino acids” refer to all naturally occuring L-α-amino acids. The amino acids are identified as hereinafter described under section A. Antibody Preparation: (iv) Generation of mutant antibodies. The term “amino acid variant” refers to molecules with some differences in their amino acid sequences as compared to a native amino acid sequence.

“Substitutional” variants are those that have at least one amino acid residue in a native sequence removed and a different amino acid inserted in its place at the same position. The substitutions may be single, where only one amino acid in the molecule as been substituted, or they may be multiple, where two or more amino acids have been substituted in the same molecule. “Insertional” variants are those with one or more amino acids inserted immediately adjacent to an amino acid at a particular position in a native sequence. Immediately adjacent to an amino acid means connected to either the α-carboxyl or α-amino functional group of the amino acid. “Deletional” variants are those with one or more amino acids in the native amino acid sequence removed. Ordinarily, deletional variants will have one or two amino acids deleted in a particular region of the molecule.

As used herein, anti-human IgE antibody means an antibody which binds to human IgE in such a manner so as to inhibit or substantially reduce the binding of such IgE to the high affinity receptor, FccRI. Preferably this anti-IgE antibody is E-25.

As used herein, the term “IgE-mediated disorder” means a condition or disease which is characterized by the overproduction and/or hypersensitivity to the immunoglobulin IgE. Specifically it should be construed to include conditions associated with anaphylactic hypersensitivity and atopic allergies, including for example: asthma, allergic rhinitis and conjunctivitis (hay fever), eczema, urticaria and food allergies.

Preferably, the anti-IgE antibody is capable of reducing or preventing any IgE-mediated disorder. The anti-IgE antibody can be substantially identical to the anti-IgE antibody E-25 and capable of binding IgE and is capable of reducing or preventing any IgE-mediated disorder. The anti-IgE antibody can be a mutant of an anti-IgE antibody, such as E-25, and capable of binding IgE and is capable of reducing or preventing any IgE-mediated disorder. The mutant can comprise a substitutional, insertional and/or deletional variant. Preferably, the anti-IgE antibody is humanized. Preferably, the anti-IgE antibody is isolated. The anti-IgE antibody can be a polyclonal antibody or monoclonal antibody. The anti-IgE antibody can be a functional fragment or analog of a anti-IgE antibody.

A preferred second active agent is omalizumab, which is a recombinant DNA-derived humanized IgG1κ monoclonal antibody that selectively binds to human IgE (Xolair®). The antibody has a molecular weight of approximately 149 kilodaltons. Xolair is produced by a Chinese hamster ovary cell suspension culture in a nutrient medium containing the antibiotic gentamicin. Gentamicin is not detectable in the final product.

Omalizumab is a sterile, white, preservative-free, lyophilized powder contained in a single-use vial that is reconstituted with Sterile Water for Injection (SWFI), USP, and administered as a subcutaneous (SC) injection. A vial of Xolair® contains 202.5 mg of omalizumab, 145.5 mg sucrose, 2.8 mg L-histidine hydrochloride monohydrate, 1.8 mg L-histdine, and 0.5 mg polysorbate 20, and is designed to deliver 150 mg of omalizumab in 1.2 mL after reconstitution with 1.4 mL SWFI, USP.

Omalizumab inhibits the binding of IgE to the high-affinity IgE receptor (FcεR1) on the surface of mast cells and basophils. Reduction in surface-bound IgE on FcεRI-bearing cells limits the degree of release of mediators of the allergic response. Treatment with omalizumab also reduces the number of FcεRI receptors on basophils in atopic patients.

Xolair® is indicated for adults and adolescents (12 years of age and above) with moderate to severe persistent asthma who have a positive skin test or in vitro reactivity to a perennial aeroallergen and whose symptoms are inadequately controlled with inhaled corticosteroids. Omalizumab has been shown to decrease the incidence of asthma exacerbations in these patients. The preferred dosage for xolair (omalizumab) is 150 to 375 mg administered subcutaneously every 2 or 4 weeks.

Asthma is characterized by a complex inflammatory response to antigens. Soluble mediators and inflammatory cells contribute to the symptoms and the airway obstruction in asthma. Medications from different therapeutic classes have been developed to antagonize specific parts of the inflammatory cascade. IgE is an immunoglobulin produced after exposure to allergens that have two forms, soluble and bound. IgE is very specific to a given allergen. It is pre-formed and therefore immediately available to bind to its receptor on primed effector cells, such as basophils and mast cells. Formation of a complex of many bound IgE-IgE receptors (‘capping’) on an effector cell is a potent stimulus for release of many pro-inflammatory mediators (histamine, leukotrienes, bradykinin, platelet activating factor etc.) The remarkable attributes of the IgE immune response is its immediacy and its specificity. Within seconds to minutes after exposure to a unique allergen, mast cells and basophils will degranulate, releasing their mediators and causing prompt itching, sneezing, swelling, bronchospasm, cough and mucus secretion. One can document this series of events by monitoring the degree of airway obstruction with spirometry. The typical response after exposure to allergen is a biphasic response with severe reductions in airflow occurring within minutes after exposure (early asthmatic response) that recovers to near normal by 1-2 hours and a late asthmatic response that occurs from six to 24 hours later. The early asthmatic response is believed to be caused by the immediate release of mediators such as those triggered by IgE. The early asthmatic response is most amenable to bronchodilators and to therapy with anti-IgE antibody. The later asthmatic response is due to inflammatory cellular influx and is amenable to therapy with glucocoticoid therapy. Fahy et al (Am J Respir Crit Care Med, 1997; 155:1828) have shown that anti-IgE antibody therapy is effective in reducing both the early (by 60%) and the late phase asthmatic response by approximately 50%. We have shown that DHEA-S is effective in reducing the late asthmatic response by 33% in patients after five days of dosing. Not intending to be limited to one mechanism of action, the combination of DHEA-S with anti-IgE therapy would have an additive effect on the late asthmatic response because both compounds affect different pathways of the allergic response. In addition to DHEA-S, other suitable non-glucocorticoid steroids can be used as the first active agent, including, but not limited to, epiandrosterone and derivative, analogs, and pharmaceutically acceptable salts thereof. For example, such as the compounds depicted by Formulas I, III, and IV, herein.

The first and second active agents are used to treat respiratory and lung diseases, and any of the additional agents listed below, may be administered per se or in the form of pharmaceutically acceptable salts, as discussed above, all being referred to as “active compounds or agents”. The first and second active agents may also be administered in combination with one another, in the form of separate, or jointly in, pharmaceutically or veterinarily acceptable formulation(s). The active compounds or their salts may be administered either systemically or topically, as discussed below.

The present invention also provides for methods for treating asthma, COPD, or other respiratory diseases comprising administering the composition to a subject in need of such treatment. The method is for prophylactic or therapeutic purposes. The method comprises an in vivo method. The method is effective for treating a plurality of diseases, whatever their cause, including steroid administration, abnormalities in adenosine or adenosine receptor metabolism or synthesis, or any other cause. The method comprises treating respiratory and lung diseases, whether by reducing adenosine or adenosine receptor levels, reducing hypersensitivity to adenosine, or any other mechanism, particularly in the lung, liver, heart and brain, or any organ that is need of such treatment. Other respiratory diseases referred to herein include cystic fibrosis (CF), dyspnea, emphysema, wheezing, pulmonary hypertension, pulmonary fibrosis, lung cancer, hyper-responsive airways, increased adenosine or adenosine receptor levels, particularly those associated with infectious diseases, pulmonary bronchoconstriction, lung inflammation, lung allergies, surfactant depletion, chronic bronchitis, bronchoconstriction, difficult breathing, impeded and obstructed lung airways, adenosine test for cardiac function, pulmonary vasoconstriction, impeded respiration, Acute Respiratory Distress Syndrome (ARDS), administration of certain drugs, such as adenosine and adenosine level increasing drugs, and other drugs for, e.g. treating SupraVentricular Tachycardia (SVT), and the administration of adenosine stress tests, infantile Respiratory Distress Syndrome (infantile RDS), pain, allergic rhinitis, decreased lung surfactant, severe acute respiratory syndrome (SARS), among others.

In one embodiment, the invention is a method for the prophylaxis or treatment of asthma comprising administering the composition to a subject in need of such treatment an amount of the composition sufficient for the prophylaxis or treatment of asthma in the subject.

In one embodiment, the invention is a method for the prophylaxis or treatment of COPD comprising administering the composition to a subject in need of such treatment an amount of the composition sufficient for the prophylaxis or treatment of COPD in the subject.

In one embodiment, the invention is a method for the prophylaxis or treatment of bronchoconstriction, lung inflammation or lung allergy comprising administering the composition to a subject in need of such treatment an amount of the composition sufficient for the prophylaxis or treatment of bronchoconstriction, lung inflammation or lung allergy in the subject.

In one embodiment, the invention is a method for the reducing or depleting adenosine in a subject's tissue comprising administering the composition to a subject in need of such treatment an amount of the composition sufficient to reduce or deplete adenosine in the subject's tissue.

The present invention also provides for a use of the first active agent and the second active agent in the manufacture of a medicament for the treatment of asthma, COPD, or other respiratory diseases, including lung cancer. The medicament comprises the composition described throughout this disclosure.

The daily dosage of the first active agent and the second active agent to be administered to a subject will vary with the overall treatment programmed, the first active agent and the second active agent to be employed, the type of formulation, the route of administration and the state of the patient. Examples 11 to 14 show aerosolized preparations in accordance with the invention for delivery with a device for respiratory or nasal administration, or administration by inhalation. For intrapulmonary administration, liquid preparations are preferred. In the case of other bioactive agents, there exist FDA recommended amounts for supplementing a person's dietary intake with additional bioactive agents, such as in the case of vitamins and minerals. However, where employed for the treatment of specific conditions or for improving the immune response of a subject they may be utilized in dosages hundreds and thousands of times higher. Mostly, the pharmacopeia's recommendations cover a very broad range of dosages, from which the medical artisan may draw guidance. Amounts for the exemplary agenis described in this patent may be in the range of those currently being recommended for daily consumption, below or above those levels. The treatment may typically begin with a low dose of a bronchodilator in combination with a non-glucocorticoid steroid, or other bioactive agents as appropriate, and then a titration up of the dosage for each patient. Higher and smaller amounts, including initial amounts, however, may be administered within the confines of this invention as well.

Preferable ranges for the first and second active agents, or any other therapeutic agent, employed here will vary depending on the route of administration and type of formulation employed, as an artisan will appreciate and manufacture in accordance with known procedures and components. The active compounds may be administered as one dose (once a day) or in several doses (several times a day). The compositions and method of preventing and treating respiratory, cardiac, and cardiovascular diseases may be used to treat adults and infants, as well as non-human animals afflicted with the described conditions. Although the present invention is concerned primarily with the treatment of human subjects, it may also be employed, for veterinary purposes in the treatment of non-human mammalian subjects, such as dogs and cats as well as for large domestic and wild animals. The terms “high” and “low” levels of “adenosine” and “adenosine receptors” as well as “adenosine depletion” are intended to encompass both, conditions where adenosine levels are higher than, or lower (even depleted) when compared to previous adenosine levels in the same subject, and conditions where adenosine levels are within the normal range but, because of some other condition or alteration in that patient, a therapeutic benefit would be achieved in the patient by decreasing or increasing adenosine or adenosine receptor levels or hypersensitivity. Thus, this treatment helps regulate (titrate) the patient in a custom tailored manner. Whereas the administration of the first active agent may decrease or even deplete adenosine levels in a subject having either normal or high levels prior to treatment, the further administration of the second active agent will improve the subject's respiration in a short period of time. The further addition of other therapeutic agents will help titrate undesirably low levels of adenosine, which may be observed upon the administration of the present treatment, particularly until an optimal titration of the appropriate dosages is attained.

Other therapeutic agents that may be incorporated into the present composition are one or more of a variety of therapeutic agents that are administered to humans and animals.

The composition can further comprise, in addition to the first and second active agents, a ubiquinone and/or folinic acid. A ubiquinone is a compound represented by the formula: 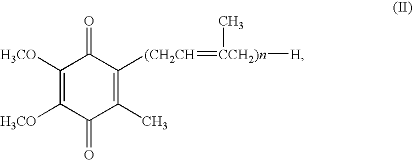
or pharmaceutically acceptable salt thereof.

Preferably, the ubiquinone is a compound according to the chemical formula given above, wherein n=1-10 (Coenzymes Q₆₋₁₀), more preferably n=6-10, (Coenzymes Q₆₋₁₀) and most preferably n=10 (Coenzyme Q₁₀). The ubiquinone is administered in a therapeutic amount for treating the targeted disease or condition, and the dosage will vary depending upon the condition of the subject, other agents being administered, the type of formulation employed, and the route of administration. The ubiquinone is preferably administered in a total amount per day of about 0.1, about 1, about 3, about 5, about 10, about 15, about 30 to about 50, about 100, about 150, about 300, about 600, about 900, about 1200 mg/kg body weight. More preferred the total amount per day is about 1 to about 150 mg/kg, about 30 to about 100 mg/kg, and most preferred about 5 to about 50 mg/kg. Ubiquinone is a naturally occurring substance and is available commercially.

The active agents of this invention are provided within broad amounts of the composition. For example, the active agents may be contained in the composition in amounts of about 0.001%, about 1%, about 2%, about 5%, about 10%, about 20%, about 40%, about 90%, about 98%, about 99.999% of the composition. The amount of each active agent may be adjusted when, and if, additional agents with overlapping activities are included as discussed in this patent. The dosage of the active compounds, however, may vary depending on age, weight, and condition of the subject. Treatment may be initiated with a small dosage, e.g. less than the optimal dose, of the first active agent of the invention. This may be similarly done with the second active agent, until a desirable level is attained. Or vice versa, for example in the case of multivitamins and/or minerals, the subject may be stabilized at a desired level of these products and then administered the first active compound. The dose may be increased until a desired and/or optimal effect under the circumstances is reached. In general, the active agent is preferably administered at a concentration that will afford effective results without causing any unduly harmful or deleterious side effects, and may be administered either as a single unit dose, or if desired in convenient subunits administered at suitable times throughout the day. The second therapeutic or diagnostic agent(s) is (are) administered in amounts which are known in the art to be effective for the intended application. In cases where the second agent has an overlapping activity with the principal agent, the dose of one of the other or of both agents may be adjusted to attain a desirable effect without exceeding a dose range that avoids untoward side effects. Thus, for example, when other analgesic and anti-inflammatory agents are added to the composition, they may be added in amounts known in the art for their intended application or in doses somewhat lower that when administered by themselves.

Pharmaceutically acceptable salts should be pharmacologically and pharmaceutically or veterinarily acceptable, and may be prepared as alkaline metal or alkaline earth salts, such as sodium, potassium or calcium salts. Organic salts and esters are also suitable for use with this invention. The active compounds are preferably administered to the subject as a pharmaceutical or veterinary composition, which includes systemic and topical formulations. Among these, preferred are formulations suitable for inhalation, or for respirable, buccal, oral, rectal, vaginal, nasal, intrapulmonary, ophthalmic, optical, intracavitary, intratraccheal, intraorgan, topical (including buccal, sublingual, dermal and intraocular), parenteral (including subcutaneous, intradermal, intramuscular, intravenous and intraarticular) and transdermal administration, among others.

The present invention also provides for a kit comprising the composition and a delivery device. The compositions may conveniently be presented in single or multiple unit dosage forms as well as in bulk, and may be prepared by any of the methods which are well known in the art of pharmacy. The composition, found in the kit, whether already formulated together or where the first and second active agents are separately provided along with other ingredients, and instructions for its formulation and administration regime. The kit may also contain other agents, such as those described in this patent and, for example, when for parenteral administration, they may be provided with a carrier in a separate container, where the carrier may be sterile. The present composition may also be provided in lyophilized form, and in a separate container, which may be sterile, for addition of a liquid carrier prior to administration. See, e.g. U.S. Pat. No. 4,956,355; UK Patent No. 2,240,472; EPO Patent Application Serial No. 429,187; PCT Patent Publication WO 91/04030; Mortensen, S. A., et al., Int. J. Tiss. Reac. XII(3): 155-162 (1990); Greenberg, S. et al., J. Clin. Pharm. 30: 596-608 (1990); Folkers, K., et al., Proc. Natl. Acad. Sci. USA 87: 8931-8934 (1990), the relevant preparatory and compound portions of which are incorporated by reference above.

The present composition is provided in a variety of systemic and topical formulations. The systemic or topical formulations of the invention are selected from the group consisting of oral, intrabuccal, intrapulmonary, rectal, intrauterine, intradermal, topical, dermal, parenteral, intratumor, intracranial, intrapulmonary, buccal, sublingual, nasal, subcutaneous, intravascular, intrathecal, inhalable, respirable, intraarticular, intracavitary, implantable, transdermal, iontophoretic, intraocular, ophthalmic, vaginal, optical, intravenous, intramuscular, intraglandular, intraorgan, intralymphatic, slow release and enteric coating formulations. The actual preparation and compounding of these different formulations is known in the art and need not be detailed here. The composition may be administered once or several times a day.

Formulations suitable for respiratory, nasal, intrapulmonary, and inhalation administration are preferred, as are topical, oral and parenteral formulations. All methods of preparation include the step of bringing the active compound into association with a carrier which constitutes one or more accessory ingredients. In general, the formulations are prepared by uniformly and intimately bringing the active compound into association with a liquid carrier, a finely divided solid carrier, or both, and then, if necessary, shaping the product into desired formulations.

Compositions suitable for oral administration may be presented in discrete units, such as capsules, cachets, lozenges, or tablets, each containing a predetermined amount of the active compound; as a powder or granules, as a solution or a suspension in an aqueous or non-aqueous liquid; or as an oil-in-water or water-in-oil emulsion.

Compositions suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the compositions isotonic with the blood of the intended recipient. Aqueous and non-aqueous sterile suspensions may include suspending agents and thickening agents. The compositions may be presented in unit-dose or multi-dose containers, for example sealed ampoules and vials, and may be stored in a freeze-dried or lyophilized condition requiring only the addition of the sterile liquid carrier, for example, saline or water-for-injection immediately prior to use.

Nasal and instillable formulations comprise purified aqueous solutions of the active compound with preservative agents and isotonic agents. Such formulations are preferably adjusted to a pH and isotonic state compatible with the nasal mucous membranes.

Formulations for rectal or vaginal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye. Otical formulations are generally prepared in viscous carriers, such as oils and the like, as is known in the art, so that they may be easily administered into the ear without spilling.

Compositions suitable for topical application to the skin preferably take the form of an ointment, cream, lotion, paste, gel, spray, aerosol, or oil. Carriers which may be used include Vaseline, lanolin, polyethylene glycols, alcohols, transdermal enhancers, and combinations of two or more thereof. Compositions suitable for transdermal administration may be presented as discrete patches adapted to remain in intimate contact with the epidermis of the recipient for a prolonged period of time.

The first and second active agents disclosed herein may be administered into the respiratory system either by inhalation, respiration, nasal administration or intrapulmonary instillation (into the lungs) of a subject by any suitable means, and are preferably administered by generating an aerosol or spray comprised of powdered or liquid nasal, intrapulmonary, respirable or inhalable particles. The respirable or inhalable particles comprising the active compound are inhaled by the subject, i.e, by inhalation or by nasal administration or by instillation into the respiratory tract or the lung itself. The formulation may comprise respirable or inhalable liquid or solid particles of the active compound that, in accordance with the present invention, include respirable or inhalable particles of a size sufficiently small to pass through the mouth and larynx upon inhalation and continue into the bronchi and alveoli of the lungs. In general, particles ranging from about 0.05, about 0.1, about 0.5, about 1, about 2 to about 4, about 6, about 8, about 10 μm in diameter. More particularly, about 0.5 to less than about 5 μm in diameter, are respirable or inhalable. Particles of non-respirable size which are included in an aerosol or spray tend to deposit in the throat and be swallowed. The quantity of non-respirable particles in the aerosol is, thus, preferably minimized. For nasal administration or intrapulmonary instillation, a particle size in the range of about 8, about 10, about 20, about 25 to about 35, about 50, about 100, about 150, about 250, about 500 μm (diameter) is preferred to ensure retention in the nasal cavity or for instillation and direct deposition into the lung. Liquid formulations may be squirted into the respiratory tract (nose) and the lung, particularly when administered to newborns and infants.

Liquid pharmaceutical compositions of active compound for producing an aerosol may be prepared by combining the active compound with a stable vehicle, such as sterile pyrogen free water. Solid particulate compositions containing respirable dry particles of micronized active compound may be prepared by grinding dry active compound with a mortar and pestle, and then passing the micronized composition through a 400 mesh screen to break up or separate out large agglomerates. A solid particulate composition comprised of the active compound may optionally contain a dispersant that serves to facilitate the formation of an aerosol. A suitable dispersant is lactose, which may be blended with the active compound in any suitable ratio, e.g., a 1 to 1 ratio by weight. The U.S. patent application Ser. Nos. 10/462,901 and 10/462,927 disclose a stable dry powder formulation of DHEA in a nebulizable form and a stable dry powder formulation of dihydrate crystal form of DHEA-S, respectively (these patent applications are herein incorporated by reference in their entirety).

Aerosols of liquid particles comprising the active compound may be produced by any suitable means, such as with a nebulizer. See, e.g. U.S. Pat. No. 4,501,729 (the disclosure of which is incorporated herein by reference). Nebulizers are commercially available devices which transform solutions or suspensions of the active ingredient into a therapeutic aerosol mist either by means of acceleration of a compressed gas, typically air or oxygen, through a narrow venturi orifice or by means of ultrasonic agitation. Suitable compositions for use in nebulizer consist of the active ingredient in liquid carrier, the active ingredient comprising up to 40% w/w composition, but preferably less than 20% w/w carrier being typically water or a dilute aqueous alcoholic solution, preferably made isotonic with body fluids by the addition of, for example sodium chloride. Optional additives include preservatives if the composition is not prepared sterile, for example, methyl hydroxybenzoate, anti-oxidants, flavoring agents, volatile oils, buffering agents and surfactants. Aerosols of solid particles comprising the active compound may likewise be produced with any sold particulate medicament aerosol generator. Aerosol generators for administering solid particulate medicaments to a subject product particles which are respirable, as explained above, and generate a volume of aerosol containing a predetermined metered dose of a medicament at a rate suitable for human administration. Examples of such aerosol generators include metered dose inhalers and insufflators.

The composition may be delivered with any delivery device that generates liquid or solid particulate aerosols, such as aerosol or spray generators. These devices produce respirable particles, as explained above, and generate a volume of aerosol or spray containing a predetermined metered dose of a medicament at a rate suitable for human or animal administration. One illustrative type of solid particulate aerosol or spray generator is an insufflator, which are suitable for administration of finely comminuted powders. In the insufflator, the powder, e.g. a metered dose of the composition effective to carry out the treatments described herein, is contained in a capsule or a cartridge. These capsules or cartridges are typically made of gelatin, foil or plastic, and may be pierced or opened in situ, and the powder delivered by air drawn through the device upon inhalation or by means of a manually-operated pump. The composition employed in the insufflator may consist either solely of the first and second agents or of a powder blend comprising the first and second agents, typically comprising from 0.01 to 100% w/w of the composition. The composition generally contains the first and second agents in an amount of about 0.01% w/w, about 1% w/w/, about 5% w/w, to about 20%, w/w/, about 40% w/w, about 99.99% w/w. Other ingredients, and other amounts of the agent, however, are also suitable within the confines of this invention.

In one embodiment, the composition is delivered by a nebulizer. This means is especially useful for patients or subjects who are unable to inhale or respire the composition under their own efforts. In serious cases, the patients or subjects are kept alive through artificial respirator. The nebulizer can use any pharmaceutically or veterinarily acceptable carrier, such as a weak saline solution. The nebulizer is the means by which the powder pharmaceutical composition is delivered to the target of the patients or subjects in the airways.

The composition is also provided in various forms that are tailored for different methods of administration and routes of delivery. In one embodiment, the composition comprises a respirable formulation, such as an aerosol or spray. The composition of the invention is provided in bulk, and in unit form, as well as in the form of an implant, a capsule, blister or cartridge, which may be openable or piercable as is known in the art. A kit is also provided, that comprises a delivery device, and in separate containers, the composition of the invention, and optionally other excipient and therapeutic agents, and instructions for the use of the kit components.

In one embodiment, the composition is delivered using suspension metered dose inhalation (MDI) formulation. Such a MDI formulation can be delivered using a delivery device using a propellant such as hydrofluroalkane (HFA). Preferably, the HFA propellants contain 100 parts per million (PPM) or less of water.

In one embodiment, the delivery device comprises a dry powder inhalator (DPI) that delivers single or multiple doses of the composition. The single dose inhalator may be provided as a disposable kit which is sterilely preloaded with enough formulation for one application. The inhalator may be provided as a pressurized inhalator, and the formulation in a piercable or openable capsule or cartridge. The kit may optionally also comprise in a separate container an agent such as other therapeutic compounds, excipients, surfactants (intended as therapeutic agents as well as formulation ingredients), antioxidants, flavoring and coloring agents, fillers, volatile oils, buffering agents, dispersants, surfactants, antioxidants, flavoring agents, bulking agents, propellants and preservatives, among other suitable additives for the different formulations.

Having now generally described this invention, the same will be better understood by reference to certain specific examples, which are included herein for purposes of illustration only and are not intended to be limiting of the invention or any embodiment thereof, unless so specified.

### EXAMPLES

### Examples 1 and 2

### In Vivo Effects of Folinic Acid and DHEA on Adenosine Levels

Young adult male Fischer 344 rats (120 grams) were administered dehydroepiandrosterone (DHEA) (300 mg/kg) or methyltestosterone (40 mg/kg) in carboxymethylcellulose by gavage once daily for fourteen days. Folinic acid (50 mg/kg) was administered intraperitoneally once daily for fourteen days. On the fifteenth day, the animals were sacrificed by microwave pulse (1.33 kilowatts, 2450 megahertz, 6.5 seconds (s)) to the cranium, which instantly denatures all brain protein and prevents further metabolism of adenosine. Hearts were removed from animals and flash frozen in liquid nitrogen with 10 s of death. Liver and lungs were removed en bloc and flash frozen with 30 s of death. Brain tissue was subsequently dissected. Tissue adenosine was extracted, derivatized to 1, N6-ethenoadenosine and analyzed by high performance liquid chromatography (HPLC) using spectrofluorometric detection according to the method of Clark and Dar (J. of Neuroscience Methods 25:243 (1988)). Results of these experiments are summarized in Table 1 below. Results are expressed as the mean±SEM, with κp<0.05 compared to control group and ψp<0.05 compared to DHEA or methyltestosterone-treated groups.

TABLE 1
In vivo Effect of DHEA, δ-1-methyltestosterone and Folinic
Acid on Adenosine Levels in various Rat Tissues

| | Intracellular adenosine (nmols)/mg protein | | | |
|---|---|---|---|---|
| Treatment | Heart | Liver | Lung | Brain |
| Control | 10.6 ± 0.6 | 14.5 ± 1.0 | 3.1 ± 0.2 | 0.5 ± 0.04 |
| | (n = 12) κ | (n = 12) κ | (n = 6) κ | (n = 12) κ |
| DHEA | 6.7 ± 0.5 | 16.4 ± 1.4 | 2.3 ± 0.3 | 0.19 ± 0.01 |
| (300 mg/kg) | (n = 12) κ | (n = 12) κ | (n = 6) κ | (n = 12) κ |
| Methyl- | 8.3 ± 1.0 | 16.5 ± 0.9 | N.D. | 0.42 ± 0.06 |
| testosterone | (n = 6) κ | (n = 6) κ | | (n = 6) κ |
| (40 mg/kg) | | | | |
| Methyl- | 6.0 ± 0.4 | 5.1 ± 0.5 | N.D. | 0.32 ± 0.03 |
| testosterone | (n = 6) κ | (n = 6) κ | | (n = 6) κ |
| (120 mg/kg) | | | | |
| Folinic Acid | 12.4 ± 2.1 | 16.4 ± 2.4 | N.D. | 0.72 ± 0.09 |
| (50 mg/kg) | (n = 5) κ | (n = 5) κ | | (n = 5) κ |
| DHEA | 11.1 ± 0.6 | 18.8 ± 1.5 | N.D. | 0.55 ± 0.09 |
| (300 mg/kg) + | (n = 5) Ψ | (n = 5) Ψ | | (n = 5) Ψ |
| Folinic Acid | | | | |
| (50 mg/kg) | | | | |
| Methyl- | 9.1 ± 0.4 | N.D. | N.D. | 0.60 ± 0.06 |
| testosterone | (n = 6) Ψ | | | (n = 6) Ψ |
| (120 mg/kg) + | | | | |
| Folinic Acid | | | | |
| (50 mg/kg) | | | | |

N.D. = Not determined

The results of these experiments indicate that rats administered DHEA or methyltestosterone daily for two weeks showed multi-organ depletion of adenosine. Depletion was dramatic in brain (60% depletion for DHEA, 34% for high dose methyltestosterone) and heart (37% depletion for DHEA, 22% depletion for high dose methyltestosterone). Coadministration of folinic acid completely abrogated steroid-mediated adenosine depletion. Folinic acid administered alone induce increase in adenosine levels for all organs studied.

### Example 3

### Airjet Milling of Anhydrous DHEA-S and Determination of Respirable Dose

DHEA-S is evaluated as an asthma therapy. The solid-state stability of sodium dehydroepiandrostenone sulfate (NaDHEA-S) has been studied for both bulk and milled material (Nakagawa, H., Yoshiteru, T., and Fujimoto, Y. (1981) Chem. Pharm. Bull. 29(5) 1466-1469; Nakagawa, H., Yoshiteru, T., and Sugimoto, I. (1982) Chem. Pharm. Bull. 30(1) 242-248). DHEA-S is most stable and crystalline as the dihydrate form. The DHEA-S anhydrous form has low crystallinity and is very hygroscopic. The DHEA-S anhydrous form is stable as long as it picks up no water on storage. Keeping a partially crystalline material free of moisture requires specialized manufacturing and packing technology. For a robust product, minimizing sensitivity to moisture is essential during the development process.

(1) Micronization of DHEA-S

Anhydrous DHEA-S was micronized using a jet milling (Jet-O-Mizer Series #00, 100-120 PSI nitrogen). Approximately 1 g sample was passed through the jet mill, once, and approximately 2 g sample were passed through the jet mill twice. The particles from each milling run were suspended in hexane, in which DHEA-S was insoluble and Spa85 surfactant added to prevent agglomeration. The resulting solution was sonicated for 3 minutes and appeared fully dispersed. The dispersed solutions were tested on a Malvern Mastersizer X with a small volume sampler (SVS) attachment. One sample of dispersed material was tested 5 times. The median particle size or D (v, 0.5) of unmilled material was 52.56 μm and the % RSD (relative standard deviation) was 7.61 for the 5 values. The D (v, 0.5) for a single pass through the jet mill was 3.90 μm and the % RSD was 1.27, and the D (v, 0.5) from a double pass through the jet mill 3.25 μm and the % RSD was 3.10. This demonstrates that DHEA-S can be jet milled to particles of size suitable for inhalation.

(2) HPLC Analysis

Two vials (A; single-pass; 150 mg) and (B double-pass; 600 mg) of the micronized drug were available for determining drug degradation during jet milling micronization. Weighed aliquots of DHEA-S from vials A and B were compared to a standard solution of unmilled DHEA-S (10 mg/ml) in an acetonitrile-water solution (1:1). The chromatographic peak area for the HPLC assay of the unmilled drug standard solution (10 mg/ml) gave a value of 23,427. Weighed aliquots of micronized DHEA-S form vials A and B, (5 mg/ml) was prepared in an acetonitrile-water solution (1:1). The chromatographic peak areas for vials A and B were 11,979 and 11,677, respectively. Clearly, there was no detectable degradation of the drug during the jet milling micronization process.

(3) Emitted Dose Studies

DHEA-S powder was collected in Nephele tubes and assayed by HPLC. Triplicate experiments were performed at each airflow rate for each of the three dry powder inhalers tested (Rotahaler, Diskhaler and IDL's DPI devices). A Nephele tube was fitted at one end with a glass filter (Gelman Sciences, Type A/E, 25 μm), which in turn was connected to the airflow line to collect the emitted dose of the drug from the respective dry powder inhaler being tested. A silicone adapter, with an opening to receive the mouthpiece of the respective dry powder inhaler being tested at the other end of the Nephele tube was secured. A desired airflow, of 30, 60, or 90 L/min, was achieved through the Nephele tube. Each dry powder inhaler's mouthpiece was inserted then into the silicone rubber adapter, and the airflow was continued for about four s after which the tube was removed and an end-cap screwed onto the end of each tube. The end-cap of the tube not containing the filter was removed and 10 ml of an HPLC grade water-acetonitrile solution (1:1) added to the tube, the end-cap reattached, and the tube shaken for 1-2 minutes. The end-cap then was removed from the tube and the solution was transferred to a 10 ml plastic syringe fitted with a filter (Cameo 13N Syringe Filter, Nylon, 0.22 μm). An aliquot of the solution was directly filtered into an HPLC vial for later drug assay via HPLC. The emitted dose experiments were performed with micronized DHEA-S (about 12.5 or 25 mg) being placed in either a gelatin capsule (Rotahaler) or a Ventodisk blister (Diskhaler and single-dose DPI (IDL)). When the micronized DHEA-S (only vial B used), was weighed for placement into the gelatin capsule or blister, there appeared to be a few aggregates of the micronized powder. The results of the emitted dose tests conducted at an airflow rate of 30, 60 and 87.8 L/min are displayed in Tables 2. Table 2 summarizes the results for the Rotahaler experiments at 3 different flow rates, for the Diskhaler experiments at 3 different flow rates, and of the multi-dose experiments at 3 different flow rates.

TABLE 2
Emitted Dose Comparison of Three Different
Dry Powder Inhaler Devices

| | Airflow Rate | |
|---|---|---|
| Inhaler Device | (L/min) | Emitted Dose (%) |
| Rotahaler | 87.8 | 73.2, 67.1, 68.7 |
| Average | | 69.7 |
| Rotahaler (2^{nd} study) | 87.8 | 16.0, 24.5, 53.9 |
| Average | | 31.5 |
| Diskhaler | 87.8 | 65.7, 41.6, 46.5 |
| Average | | 51.3 |
| Diskhaler (2^{nd} study) | 87.8 | 57.9, 59.9, 59.5 |
| Average | | 59.1 |
| IDL Multi-Dose | 87.8 | 71.3, 79.0, 67.4 |
| Average | | 72.6 |
| IDL Multi-Dose (2^{nd} study) | 87.8 | 85.7, 84.6, 84.0 |
| Average | | 84.8 |
| Rotahaler | 60 | 58.1, 68.2, 45.7 |
| Average | | 57.3 |
| Diskhaler | 60 | 63.4, 38.9, 58.0 |
| Average | | 68.2 |
| IDL Multi-Dose | 60 | 78.8, 83.7, 89.6 |
| Average | | 84.0 |
| Rotahaler | 30 | 34.5, 21.2, 48.5 |
| Average | | 34.7 |
| Diskhaler | 30 | 53.8, 53.4, 68.7 |
| | | 58.6 |
| IDL Multi-Dose | 30 | 78.9, 88.2, 89.2 |
| Average | | 85.4 |

(4) Respirable Dose Studies

The respirable dose (respirable fraction) studies were performed using a standard sampler cascade impactor (Andersen), consisting of an inlet cone (an impactor pre-separator was substituted here), 9 stages, 8 collection plates, and a backup filter within 8 aluminum stages held together by 3 spring clamps and gasket O-ring seals, where each impactor stage contains multiple precision drilled orifices. When air is drawn through the sampler, multiple jets of air in each stage direct any airborne particles toward the surface of the collection plate for that stage. The size of the jets is constant for each stage, but is smaller in each succeeding stage. Whether a particle is impacted on any given stage depends upon its aerodynamic diameter. The range of particle sizes collected on each stage depends upon on the jet velocity of the stage, and the cut-off point of the previous stage. Any particle not collected on the first stage follows the air stream around the edge of the plate to the next stage, where it is either impacted or passed on to the succeeding stage, and so on, until the velocity of the jet is sufficient for impaction. To prevent particle bounce during the cascade impactor test, the individual impactor plates were coated with a hexane-grease (high vacuum) solution (100:1 ratio). As noted above, the particle size cut-off points on the impactor plates changed at different airflow rates. For example, Stage 2 corresponds to a cut-off value greater than 6.2 μm particles at 60 L/min, and greater than 5.8 μm particles at 30 L/min, and stage 3 had a particle size cut-off value at 90 L/min greater than 5.6 μnm. Thus, similar cut-off particle values are preferentially employed at comparable airflow rates, i.e. ranging from 5.6 to 6.2 μm. The set-up recommended by the United States Phamacopeia for testing dry powder inhalers consists of a mouthpiece adapter (silicone in this case) attached to a glass throat (modified 50 ml round-bottom flask) and a glass distal pharynx (induction port) leading top the pre-separator and Andersen sampler. The pre-separator sample includes washings from the mouthpiece adaptor, glass throat, distal glass pharynx and pre-separator. 5 ml acetonitrile:water (1:1 ratio) solvent was placed in the pre-separator before performing the cascade impactor experiment, that were performed in duplicate with 3 different dry powder inhaler devices and at 3 airflow rates, 30, 60 and 90 L/min. The drug collected on the cascade impactor plates were assayed by the HPLC, and a drug mass balance was performed for each Diskhaler and multi-dose cascade impactor experiment consisting of determining the amount of drug left in the blister, the amount of drug remaining in the device (Diskhaler only), the non-respirable amount of the dose retained on the silicone rubber mouth piece adaptor, glass throat, glass distal pharynx and pre-separator, all combined into one sample, and the respirable dose, i.e. Stage 2 through filter impactor plates for airflow rates of 30 and 60 L/min and Stages 1 through filter impactor plates for 90 L/min experiments.

TABLE 3
Cascade Impactor Experiments (90 L/min)

| | | | | | Mass |
|---|---|---|---|---|---|
| Inhaler | Preseparator | Blister | Respirable | Device | Balance |
| Device | (%) | (%) | Dose (%) | (%) | (%) |
| Diskhaler | 72.7 | 6.6 | 2.9 | 22.1 | 104.3 |
| Diskhaler | 60.2 | 10.1 | 2.4 | 13.3 | 86.0 |
| Multi-dose | 65.8 | 3.9 | 3.8 | 26.5 *^{a} | 100.0 |
| Multi-dose | 73.3 | 3.8 | 3.6 | 19.3 *^{a} | 100.0 |
| Multi-dose *^{b} | 78.7 | 2.8 | 4.6 | 13.9 *^{a} | 100.0 |
| Multi-dose *^{c} | 55.9 | 5.0 | 1.2 | 37.9 *^{a} | 100.0 |

*^{a} Multi-dose device was not washed; as solvents would attack SLA components. Multi- dose device retention percentage is obtained by difference.
*^{b} oven dried drug for 80 minutes
*^{c} oven dried drug for 20 hours

Based on the results of the emitted dose and cascade impactor experiments, the low respirable dose values achieved in the cascade impactor experiments were due to agglomerated drug particles, which could not be separated, even at the highest airflow rate tested. Agglomeration of the drug particles is a consequence of static charge build up during the mechanical milling process used for particles size reduction and that this situation is further compounded by subsequent moisture absorption of the particles. A micronization method that produces less static charge or a less hygroscopic, fully hydrated crystalline form of DHEA-S (i.e. dihydrate form) should provide a freer flowing powder with diminished potential for agglomeration.

### Example 4

### Spray Drying of Anhydrous DHEA-S and Determination of Respirable Dose

(1) Micronization of the Drug

1.5 g of anhydrous DHEA-S were dissolved to 100 ml of 50% ethanol:water to produce a 1.5% solution. The solution was spray-dried with a B-191 Mini Spray-Drier (Buchi, Flawil, Switzerland) with an inlet temperature of 55° C., outlet temperature of 40° C., at 100% aspirator, at 10% pump, nitrogen flow at 40 mbar and spray flow at 600 units. The spray-dried product was suspended in hexane and Span85 surfactant added to reduce agglomeration. The dispersions were sonicated with cooling for 3-5 minutes for complete dispersion and the dispersed solutions tested on a Malvern Mastersizer X with a Small Volume Sampler (SVS) attachment. The two batches of spray dried material were found to have mean particle sizes of 5.07±0.70 μm and 6.66±0.91 μm. Visual examination by light microscope of the dispersions of each batch confirmed that spray drying produced small respirable size particles. The mean particle size was 2.4 μm and 2.0 μm for each batch, respectively. This demonstrates that DHEA-S can be spray dried to a particle size suitable for inhalation.

(2) Respirable Dose Studies

The cascade impactor experiments were conducted as described in Example 3. Four cascade impactor experiments were done, three with a IDL multi-dose device and one with a Diskhaler, all at 90 L/min. The results of the cascade impactor experiments are presented in Table 4 below. The spray-dried anhydrous material in these experiments produced a two-fold increase in the respirable dose compared to micronized anhydrous DHEA-S. It appears that spray drying obtained higher respirable doses as compared to jet-milling. However, the % respirable dose was still low. This was likely the result of moisture absorption of the anhydrous form.

TABLE 4
Cascade Impactor Results with Spray-Dried Drug Product

| Device | Diskhaler | Multi-dose | Multi-dose | Multi-dose |
|---|---|---|---|---|
| Number of Blisters | 3 | 3 | 4 | 4 |
| Drug per Blister (mg) | 38.2 | 36.7 | 49.4 | 50.7 |
| Preseparator (%) | 56.8 | 71.9 | 78.3 | 85.8 |
| Device (%) | 11.2 | 7.9 | 8.9 | 7.6 |
| Blisters (%) | 29.0 | 6.4 | 8.2 | 4.8 |
| Respirable Dose (%) | 5.6 | 7.8 | 5.3 | 2.6 |
| Mass Balance | 102.7 | 94.0 | 103.3 | 98.1 |
| Recovery (%) | | | | |

### Example 5

### Air Jet Milling of DHEA-S Dihydrate (DHEA-S.2H₂O) and Determination of Respirable Dose

(1) Recrystallization of DHEA-S Dihydrate

Anhydrous DHEA-S is dissolved in a boiling mixture of 90% ethanol/water. This solution is rapidly chilled in a dry ice/methanol bath to recrystallize the DHEA-S. The crystals are filtered, washed twice with cold ethanol, than dried in a vacuum desiccator at RT for 36 h. During the drying process, the material is periodically mixed with a spatula to break large agglomerates. After drying, the material is passed through a 500 μm sieve.

(2) Micronization and Physiochecmical Testing.

DHEA-S dihydrate is micronized with nitrogen gas in a jet mill at a venturi pressure of 40 PSI, a mill pressure of 80 PSI, feed setting of 25 and a product feed rate of about 120 to 175 g/hour. Surface area is determined using five point BET analyses are performed with nitrogen as the adsorbing gas (P/Pₒ=0.05 to 0.30) using a Micromeritics TriStar surface area analyzer. Particle size distributions are measured by laser diffraction using a Micromeritics Saturn Digisizer where the particles are suspended in mineral oil with sodium dioctyl sodium sulfosuccinate as a dispersing agent. Drug substance water content is measured by Karl Fischer titration (Schott Titroline KF). Pure water is used as the standard and all relative standard deviations for triplicates are less than 1%. Powder is added directly to the titration media. The physicochemical properties of DHEA-S-dihydrate before and after micronization are summarized in Table 5.

TABLE 5
Physicochemical properties of DHEA-S · dihydrate
before and after micronization.

| Property | Bulk | Micronized |
|---|---|---|
| Particle size (D_{50%}) | 31 microns | 3.7 microns |
| Surface area (m²/g) | Not measured | 4.9 |
| Water (% w/w) | 8.5 | 8.4 |
| Impurities | No significant peaks | No significant peaks |

The only significant change measured is in the particle size. There is no significant loss of water or increase in impurities. The surface area of the micronized material is in agreement with an irregularly shaped particle having a median size of 3 to 4 microns. The micronization successfully reduces the particle size to a range suitable for inhalation with no measured changes in the solid-state chemistry.

(3) Aerosolization of DHEA-S-Dihydrate.

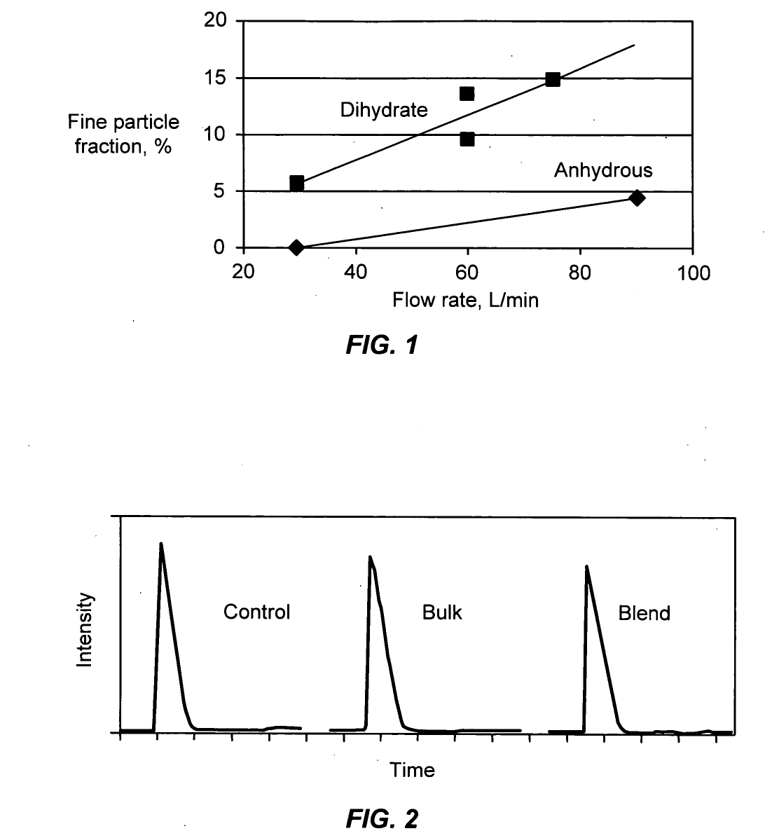

The single-dose Acu-Breathe device is used for evaluating DHEA-S-dihydrate. Approximately 10 mg of neat DHEA-S-dihydrate powder is filled and sealed into foil blisters. These blisters are actuated into the Andersen 8-stage cascade impactor at flow rates ranging from 30 to 75 L/min with a glass twin-impinger throat. Stages 1-5 of the Andersen impactor are rinsed together to obtain an estimate of the fine particle fraction. Pooling the drug collected from multiple stages into one assay make the method much more sensitive. The results for this series of experiments is shown in FIG. 1. At all flow rates, the dihydrate yields a higher fine particle fraction than the virtually anhydrous material. Since the dihydrate powder is aerosolized using the single-dose inhaler, it is very reasonable to conclude that its aerosol properties are significantly better than the virtually anhydrous material. Higher crystallinity and stable moisture content are the most likely factors contributing the dihydrate's superior aerosol properties. This unique feature of DHEA-S-dihydrate has not been reported in any previous literature. While the improvement in DHEA-S's aerosol performance with the dihydrate form is significant, neat drug substance may not be the optimal formulation. Using a carrier with a larger particle size typically improves the aerosol properties of micronized drug substances.

### Example 6

### Anhydrous DHEA-S and DHEA-S Dihydrate Stability with and without Lactose

The initial purity (Time=0) was determined for anhydrous DHEA and for DHEA-S dihydrate by high pressure liquid chromatography (HPLC). Both forms of DHEA-S were then either blended with lactose at a ratio of 50:50, or used as a neat powder and placed in open glass vials, and held at 50° C. for up to 4 weeks. These conditions were used to stress the formulation in order to predict its long-term stability results. Control vials containing only DHEA-S (anhydrous or dihydrate) were sealed and held 25° C. for up to 4 weeks. Samples were taken and analyzed by HPLC also at 0, 1, 2, and 4 weeks to determine the amount of degradation, as determined by formation of DHEA. After one week, virtually anhydrous DHEA-S blended with lactose (50% w/w, nominally) stored at 50° C. in sealed glass vials acquires a brown tinge that is darker for the lactose blend. This color change is accompanied by a significant change in the chromatogram as shown in FIG. 1. The primary degradant is DHEA. Qualitatively from FIG. 2, the amount of DHEA in the blend is higher than the other two samples. To quantitatively estimate the % DHEA in the samples, the area for the DHEA peak is divided by the total area for the DHEA-S and DHEA peaks (see Table 6). The higher rate of decomposition for the blend indicates a specific interaction between lactose and the virtually anhydrous DHEA-S. In parallel with the increase in DHEA, the brown color of the powders on accelerated storage increased over time. The materials on accelerated storage become more cohesive with time as evidenced by clumping during sample weighing for chemical analysis. Based on these results, it is not possible to formulate virtually anhydrous DHEA-S with lactose. This is a considerable disadvantage since lactose is the most commonly used inhalation excipient for dry powder formulations. Continuing with the virtually anhydrous form would mean limiting formulations to neat powder or undertaking more comprehensive safety studies to use a novel excipient.

TABLE 6
DHEA % formed from Anhydrous DHEA-S at 50° C.

| | Time | | | |
|---|---|---|---|---|
| Formulation | (Weeks) | 1 | 2 | 4 |
| Control | 2.774 | 2.694 | 2.370 | 2.666 |
| DHEA-S. Alone | | 9.817 | 14.954 | 20.171 |
| DHEA-S + Lactose | | 24.085 | 30.026 | 38.201 |
| (50:50) | | | | |

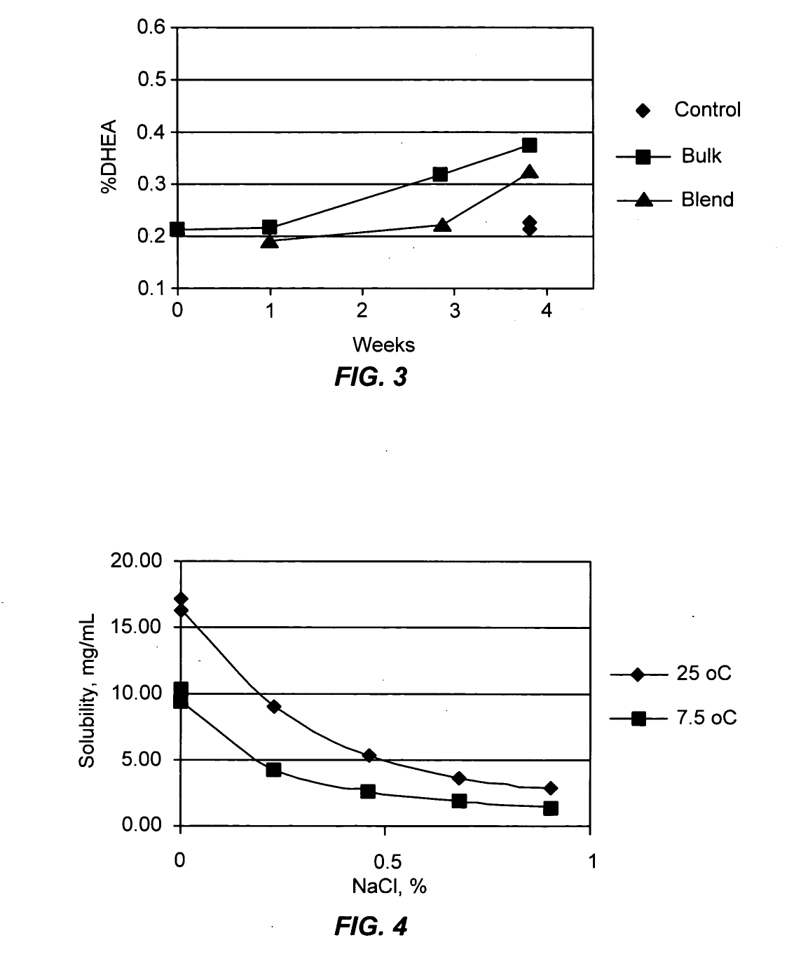

In contrast to FIG. 2, there is virtually no DHEA generated after storage for 1 week at 50° C. (see FIG. 3). Furthermore, the materials show no change in color. The moisture content of DHEA-S-dihydrate remains virtually unchanged after one week at 50° C. The water content after accelerated storage is 8.66% versus a starting value of 8.8%. The % DHEA measured during the course of this stability program is shown in Table 7.

TABLE 7
Percent DHEA formed from DHEA-S Dihydrate at 50° C.

| | Time | | | |
|---|---|---|---|---|
| Formulation | (Weeks) | 1 | 3 | 4 |
| Control | 0.213 | | | 0.218 |
| DHEA-S alone | | 0.216 | 0.317 | 0.374 |
| DHEA-S:Lactose | | 0.191 | 0.222 | 0.323 |
| (50:50) | | | | |

By comparing FIGS. 1 and 2 and Tables 6 and 7, one can see that the dihydrate form of DHEA-S is the more stable form for progression into further studies. The superior compatibility of DHEA-S-dihydrate with lactose over that of the virtually anhydrous material has not been reported in the patent or research literature. The solubility of this substance is reported in the next section as a portion of the development work for a nebulizer solution.

### Example 7

### DHEA-S Dihydrate/Lacotse Blends, Determination of Respirable Dose and Stability

(1) DHEA-S Dihydrate/Lactose Blend.

Equal weights of DHEA-S and inhalation grade lactose (Foremost Aero Flo 95) are mixed by hand then passed through a 500 μm screen to prepare a pre-blend. The pre-blend is then placed in a BelArt Micro-Mill with the remaining lactose to yield a 10% w/w blend of DHEA-S. The blender is wired to a variable voltage source to regulate the impeller speed. The blender voltage is cycled through 30%, 40%, 45% and 30% of full voltage for 1, 3, 1.5, and 1.5 minutes, respectively. The content uniformity of the blend was determined by HPLC analysis. Table 8 shows the result of content uniformity samples for this blend. The target value is 10% w/w DHEA-S. The blend content is satisfactory for proximity to the target value and content uniformity.

TABLE 8
Content uniformity for a blend of
DHEA-S · dihydrate with lactose.

| Sample | % DHEA-S, w/w |
|---|---|
| 1 | 10.2 |
| 2 | 9.7 |
| 3 | 9.9 |
| 4 | 9.3 |
| 5 | 9.4 |
| Mean | 9.7 |
| RSD | 3.6% |

(2) Aerosolization of DHEA-S-Dihydrate/Lactose Blend.

Approximately 25 mg of this powder is filled and sealed in foil blisters and aerosolized using the single-dose device at 60 L/min. Two blisters are used for each test and the results for fine particle fraction (material on stages 1-5) are shown in Table 9. The aerosol results for this preliminary powder blend are satisfactory for a respiratory drug delivery system. Higher fine particle fractions are possible with optimization of the powder blend and blister/device configuration. The entire particle size distribution of Test 2 is shown in Table 10. This median diameter for DHEA-S for this aerosol is ˜2.5 μm. This diameter is smaller than the median diameter measured for micronized DHEA-S.dihydrate by laser diffraction. Irregularly shaped particles can behave aerodynamically as smaller particles since their longest dimension tends to align with the air flow field. Therefore, it is common to see a difference between the two methods. Diffraction measurements are a quality control test for the input material while cascade impaction is a quality control test for the finished product.

TABLE 9
Fine particle fraction for lactose
blend in two different experiments

| | Total powder weight | DHEA-S collected | Fine particle |
|---|---|---|---|
| Test | in two blisters (mg) | Stages 1-5 (mg) | fraction, % |
| 1 | 52.78 | 1.60 | 31 |
| 2 | 57.09 | 1.62 | 29 |

TABLE 10
Particle size distribution of aerosolized
DHEA-S dihydrate/Lactose Blend

| | | | | | | | | |
|---|---|---|---|---|---|---|---|---|
| Size (μm) | 6.18 | 9.98 | 3.23 | 2.27 | 1.44 | 0.76 | 0.48 | 0.27 |
| % Particles | 100 | 87.55 | 67.79 | 29.87 | 10.70 | 2.57 | 1.82 | 0.90 |
| Under | | | | | | | | |

(3) Stability of DHEA-S Dihydrate/Lactose Blend.

This lactose formulation is also placed on an accelerated stability program at 50° C. The results for DHEA-S content are in Table 11. The control is the blend stored at RT. There is no trend in the DHEA-S content over time for either condition and all the results are within the range of samples collected for content uniformity testing (see Table 11). Furthermore, there are no color changes or irregularities observed in the chromatograms. The blend appears to be chemically stable.

TABLE 11
Stressed stability data on DHEA-S · dihydrate/lactose
blend at 50°C.

| | % DHEA-S w/w for control | % DHEA-S w/w for |
|---|---|---|
| Time (weeks) | condition | stressed condition |
| 0 | 9.7 | 9.7 |
| 1 | 9.6 | 9.6 |
| 1.86 | 9.5 | 9.7 |
| 3 | 10 | 9.9 |

### Example 8

### Nebulizer Formulation of DHEA-S

Solubility of DHEA-S.

An excess of DHEA-S dihydrate, prepared according to “Recrystallization of DHEA-S-Dihydrate (Example 5)”, is added to the solvent medium and allowed to equilibrate for at least 14 hours with some periodic shaking. The suspensions are then filtered through a 0.2 micron syringe filter and immediately diluted for HPLC analysis. To prepare refrigerated samples, the syringes and filters are stored in the refrigerator for at least one hour before use. Inhalation of pure water can produce a cough stimulus. Therefore, it is important to add halide ions to a nebulizer formulation with NaCl being the most commonly used salt. Since DHEA-S is a sodium salt, NaCl could decrease solubility due to the common ion effect. The solubility of DHEA-S at RT (24-26° C.) and refrigerated (7-8° C.) as a function of NaCl concentration is shown in FIG. 4. DHEA-S's solubility decrease with NaCl concentration. Lowering the storage temperature decrease the solubility at all NaCl concentrations. The temperature effect is weaker at high NaCl concentrations. For triplicates, the solubility at ˜25° C. and 0% NaCl range from 16.5-17.4 mg/mL with a relative standard deviation of 2.7%. At 0.9% NaCl refrigerated, the range for triplicates is 1.1-1.3 mg/mL with a relative standard deviation of 8.3%.

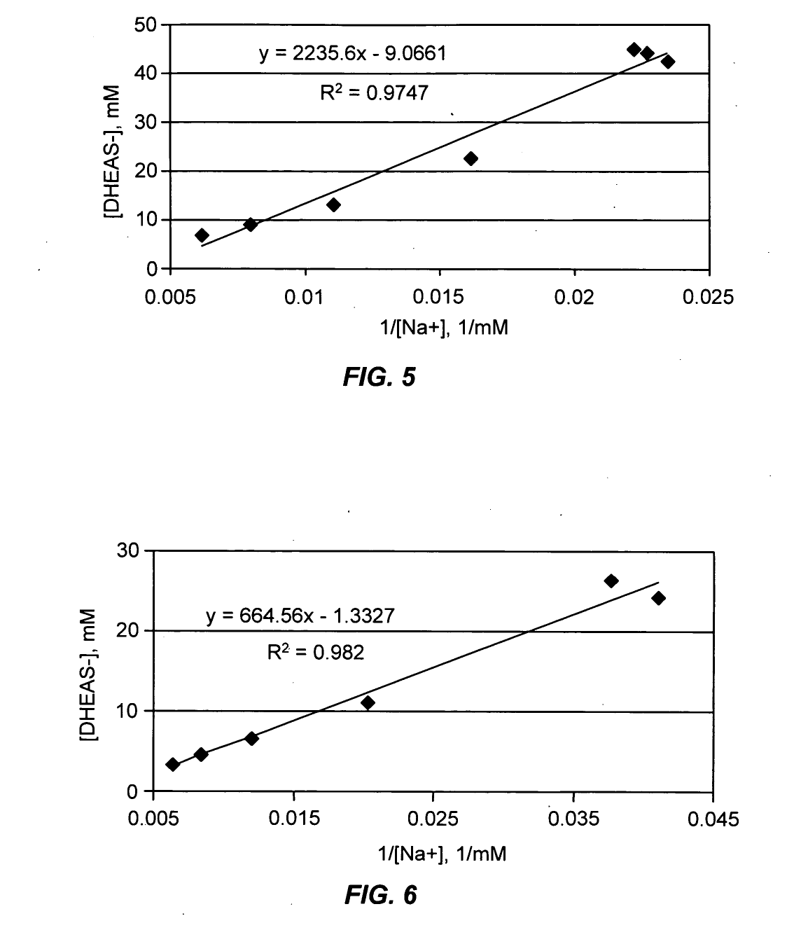

The equilibrium between DHEA-S in the solid and solution states is:
NaDHEA-S_{solid}⇄DHEA-S⁻-+Na⁺
K=[DHEA-S⁻][Na⁺]/[NaDHEA-S]_{solid}
Since the concentration of DHEA-S in the solid is constant (i.e., physically stable dihydrate), the equilibrium expression is simplified:
Ksp=[DHEA-S⁻][Na⁺]
Based on this presumption, a plot of DHEA-S solubility versus the reciprocal of the total sodium cation concentration is linear with a slope equal to Ksp. This is shown in FIGS. 5 and 6 for equilibrium at RT and refrigerated, respectively. Based on the correlation coefficients, the model is a reasonable fit to the data at both room and refrigerated temperatures where the equilibrium constants were 2236 and 665 mM², respectively. To maximize solubility, the NaCl level needs to be as low as possible. The minimum halide ion content for a nebulizer solution should be 20 mM or 0.12% NaCl.

To estimate a DHEA-S concentration for the solution, a 10° C. temperature drop in the nebulizer during use is assumed (i.e., 15° C.). Interpolating between the equilibrium constants versus the reciprocal of absolute temperature, the Ksp at 15° C. would be ˜1316 mM². Each mole of DHEA-S contributes a mole of sodium cation to the solution, therefore:

$$\begin{matrix}Ksp = [ DHEA \text{-} {S}^{-} ] [ {Na}^{+} ] \\ = [ DHEA \text{-} {S}^{-} ] [ {Na}^{+} + DHEA \text{-} {S}^{-} ] \\ = {[ DHEA \text{-} {S}^{-} ]}^{2} + [ {Na}^{+} ] [ DHEA \text{-} {S}^{-} ]\end{matrix}$$

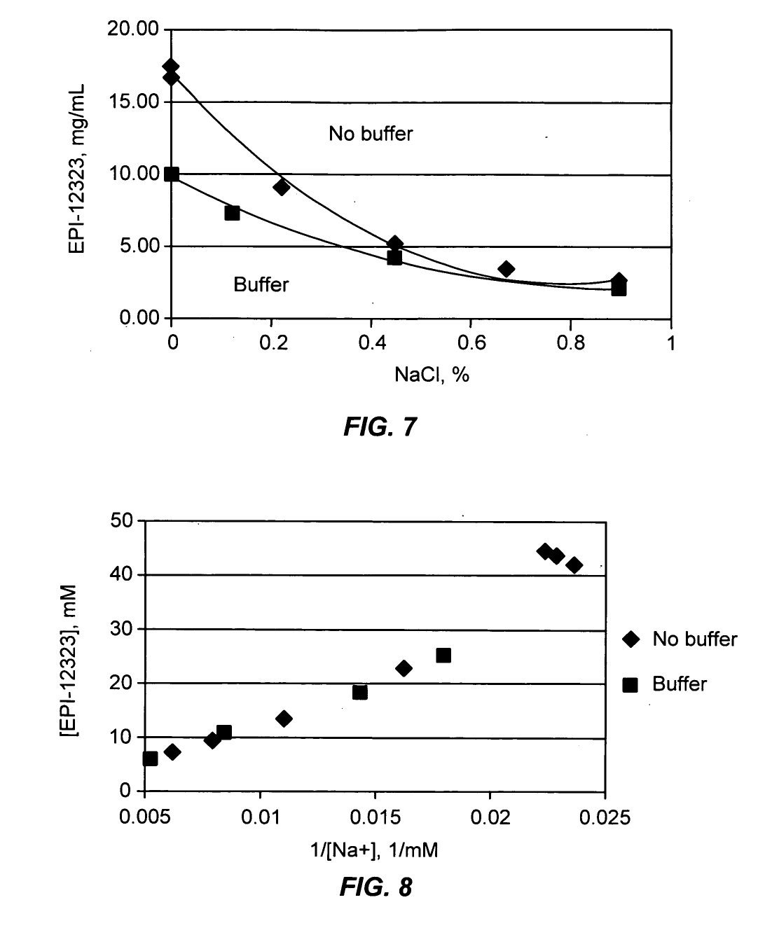

which is solve for [DHEA-S⁻] using the quadratic formula. The solution for 20 mM Na⁺ with a Ksp of 1316 mM² is 27.5 mM DHEA-S— or 10.7 mg/mL. Therefore a 10 mg/mL DHEA-S solution in 0.12% NaCl is selected as a good candidate formulation to progress into additional testing. The estimate for this formula does not account for any concentration effects due to water evaporation from the nebulizer. The pH of a 10 mg/mL DHEA-S solution with 0.12% NaCl range from 4.7 to 5.6. While this would be an acceptable pH level for an inhalation formulation, the effect of using a 20 mM phosphate buffer is evaluated. The solubility results at RT for buffered and unbuffered solutions are shown in FIG. 7. The presence of buffer in the formulation suppress the solubility, especially at low NaCl levels. As shown in FIG. 8, the solublity data for the buffered solution falls on the same equilibrium line as for the unbuffered solution. The decrease in solubility with the buffer is due to the additional sodium cation content. Maximizing solubility is an important goal and buffering the formulation reduces solubility. Furthermore, Ishihora and Sugimoto ((1979) Drug Dev. Indust. Pharm. 5(3) 263-275) did not show a significant improvement in NaDHEA-S stability at neutral pH.
Stability Studies.

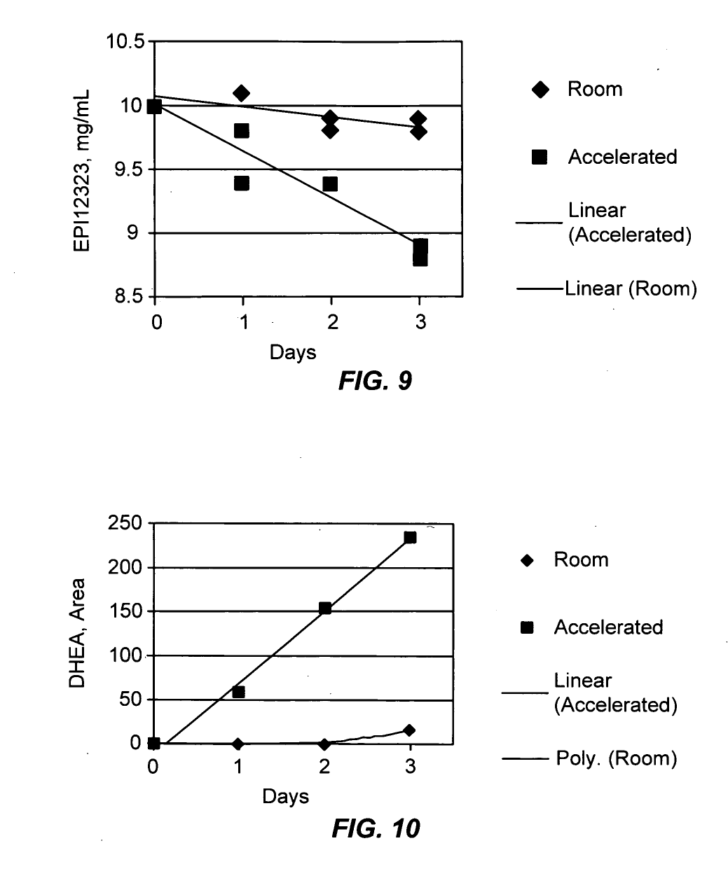

A 10 mg/mL DHEA-S formulation is prepared in 0.12% NaCl for a short-term solution stability program. Aliquots of this solution are filled into clear glass vials and stored at RT (24-26° C.) and at 40° C. The samples are checked daily for DHEA-S content, DHEA content, and appearance. For each time point, duplicate samples are withdrawn and diluted from each vial. The DHEA-S content over the length of this study is shown in FIGS. 9 and 10. At the accelerated condition, the solution show a faster decomposition rate and became cloudy after two days of storage. The solution stored at RT is more stable and a slight precipitate is observed on the third day. The study is stopped on day three. DHEA-S decomposition is accompanied by an increase in DHEA content as shown in FIG. 10. Since DHEA is insoluble in water, it only takes a small quantity in the formulation to create a cloudy solution (accelerated storage) or a crystalline precipitate (room storage). This explains why earlier visual evaluations of DHEA-S solubility severely underestimate the compound's solubility: small quantities of DHEA would lead the experimenter to conclude the solubility limit of DHEA-S had been exceeded. The solution should easily be stable for the day of reconstitution in a clinical trial. The following section describes the aerosol properties of this formulation.

Nebulizer Studies.

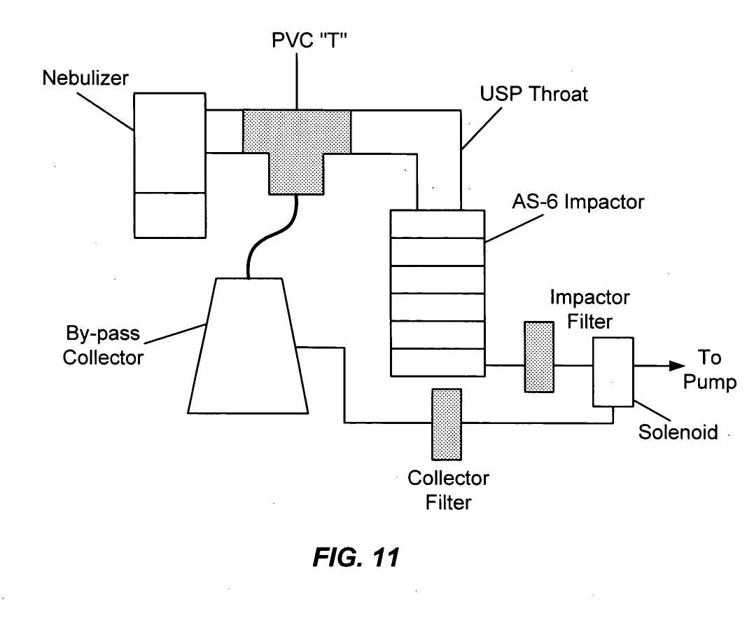

DHEA-S solutions are nebulized using a Pari ProNeb Ultra compressor and LC Plus nebulizer. The schematic for the experiment set-up is shown in FIG. 11. The nebulizer is filled with 5 mL of solution and nebulization is continued until the output became visually insignificant (4Y2 to 5 min.). Nebulizer solutions are tested using a California Instruments AS-6 6-stage impactor with a USP throat. The impactor is run at 30 L/min for 8 s to collect a sample following one minute of nebulization time. At all other times during the experiment, the aerosol is drawn through the by-pass collector at approximately 33 L/min. The collection apparatus, nebulizer, and impactor are rinsed with mobile phase and assayed by HPLC. 5 mL of DHEA-S in 0.12% NaCl is used in the nebulizer. This volume is selected as the practical upper limit for use in a clinical study. The results for the first 5 nebulization experiments are shown below:

TABLE 12
Results for nebulization studies with DHEA-S

| | Left in | | | |
|---|---|---|---|---|
| Solution- | Nebulizer, | Deposited in | Deposited in | Total, |
| Nebulizer # | mg | Collector, mg | Impactor, mg | mg |
| 10 mg/mL-1 | 17.9* | 16.3 | 0.38 | 34.6 |
| 10 mg/mL-2 | 31.2 | 17.2 | 0.48 | 49.0 |
| 7.5 mg/mL-1 | 19.3 | 16.3 | 0.35 | 36.0 |
| 7.5 mg/mL-1 | 21.7 | 15.4 | 0.30 | 37.4 |
| 5.0 mg/mL-1 | 14.4 | 10.6 | 0.21 | 25.2 |

*Only assayed liquid poured from nebulizer; did not weigh before and after aerosolization or rinse entire unit

Nebulizer #1 runs to dryness in about 5 minutes while Nebulizer #2 takes slightly less than 4.5 minutes. In each case, the liquid volume remaining in the nebulizer is approximately 2 mL. This liquid is cloudy initially after removal from the nebulizer then clears within 3-5 minutes. Even after this time, the 10 mg/mL solutions appear to have a small amount of coarse precipitate in them. Fine air bubbles in the liquid appear to cause the initial cloudiness. DHEA-S appears to be surface active (i.e., promoting foam) and this stabilizes air bubbles within the liquid. The precipitate in 10 mg/mL solutions indicates that the drug substance's solubility is exceeded in the nebulizer environment. Therefore, the additional nebulization experiments in Table 13 are run at lower concentrations. able 13 presents additional data of “dose” linearity versus solution concentration.

TABLE 13
Results from additional nebulizer experiments with DHEA-S.

| | Left in | | | |
|---|---|---|---|---|
| Solution- | Nebulizer, | Deposited in | Deposited in | Total, |
| Nebulizer # | mg | Collector, mg | Impactor, mg | mg |
| 6.25 mg/mL-2 | 17.8 | 12.1 | 0.24 | 30.1 |
| 7.5 mg/mL-3 | 21.2 | 13.8 | 0.33 | 35.3 |

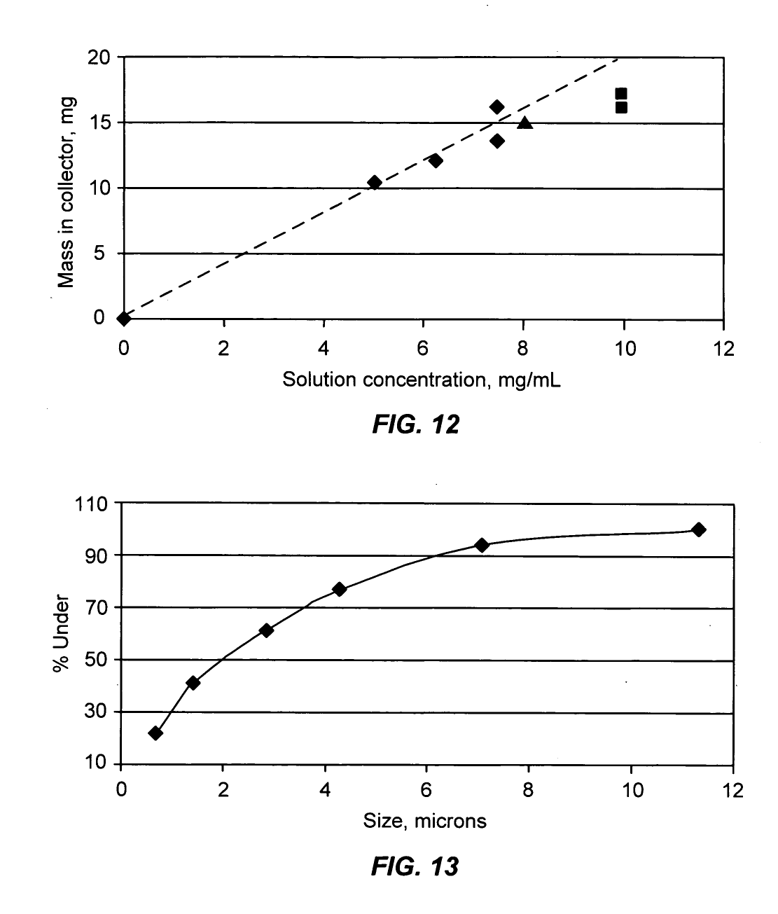

Nebulizer #3 takes slightly less than 4.5 minutes to reach dryness. The mass in the by-pass collector is plotted versus the initial solution concentration in FIG. 12. There is good linearity from 0 to 7.5 mg/mL then the amount collected appears to start leveling-off. While the solubility reduction by cooling is included in the calculation of the 10 mg/mL solution, any concentration effects on drug and NaCl content were neglected. Therefore, it is possible for a precipitate to form via supersaturation of the nebulizer liquid. The data in FIG. 12 and the observation of some particulates in the 10 mg/mL solution following nebulization indicate that the highest solution concentration for a proof of concept clinical trial formulation is approximately 7.5 mg/mL. An aerosol sample is drawn into a cascade impactor for particle size analysis. There is no detectable trend in particle size distribution with solution concentration or nebulizer number. The average particle size distribution for all nebulization experiments is shown in FIG. 13. The aerosol particle size measurements are in agreement with published/advertised results for this nebulizer (i.e., median diameter ˜2 μm). While the in vitro experiments demonstrate that a nebulizer formulation can deliver respirable DHEA-S aerosols, the formulation is unstable and takes 4-5 minutes of continuous nebulization. Therefore, a stable DPI formulation has significant advantages. DHEA-S-dihydrate is identified as the most stable solid state for a DPI formulation. An optimal nebulizer formulation is 7.5 mg/mL of DHEA-S in 0.12% NaCl for clinical trials for DHEA-S. The pH of the formulation is acceptable without a buffer system. The aqueous solubility of DHEA-S is maximized by minimizing the sodium cation concentration. Minimal sodium chloride levels without buffer achieve this goal. This is the highest drug concentration with 20 mM of Cl⁻ that will not precipitate during nebulization. This formulation is stable for at least one day at RT.

### Example 9

### Preparation of the Experimental Model

Cell cultures, HT-29 SF cells, which represent a subline of HY-29 cells (ATCC, Rockville, Md.) and are adapted for growth in completely defined serum-free PC-1 medium (Ventrex, Portland, Me.), were obtained. Stock cultures were maintained in this medium at 37° C. (in a humidified atmosphere containing 5% CO₂). At confluence cultures were replated after dissociation using trypsin/EDTA (Gibco, Grand Island, N.Y.) and re-fed every 24 hours. Under these conditions, the doubling time for HT-29 SF cells during logarithmic growth was 24 hours.

Flow Cytometry

Cells were plated at 10⁵/60-mm dish in duplicate. For analysis of cell cycle distribution, cultures were exposed to 0, 25, 50, or 200 μM DHEA. For analysis of reversal of cell cycle effects of DHEA, cultures were exposed to either 0 or 25 μM DHEA, and the media were supplemented with MVA, CH, RN, MVA plus CH, or MVA plus CH plus RN or were not supplemented. Cultures were trypsinized following 0, 24, 48, or 74 hours and fixed and stained using a modification of a procedure of Bauer et al., Cancer Res. 46, 3173-3178 (1986). Briefly, cells were collected by centrifugation and resuspended in cold phosphate-buffered saline. Cells were fixed in 70% ethanol, washed, and resuspended in phosphate-buffered saline. One ml hypotonic stain solution (50 μg/ml propidium iodide (Sigma Chemical Co.), 20 μg/ml Rnase A (Boehringer Mannheim, Indianapolis, Ind.), 30 mg/ml polyethylene glycol, 0.1% Triton X-100 in 5 mM citrate buffer) was then added, and after 10 min at room temperature, 1 ml of isotonic stain solution (propidium iodide, polyethylene glycol, Triton X-100 in 0.4M NaCl) was added and the cells were analyzed using a flow cytometer, equipped with pulse width/pulse area doublet discrimination (Becton Dickinson Immunocytometry Systems, San Jose, Calif.) After calibration with fluorescent beads, a minimum of 2×10⁴ cells/sample were analyzed, data were displayed s total number of cells in each of 1024 channels of increasing fluorescence intensity, and the resulting histogram was analyzed using the Cellfit analysis program (Becton Dickinson).

DHEA Effect on Cell Growth

Cells were plated 25,000 cells/30 mm dish in quadruplicate, and after 2 days received 0, 12.5, 25, 50, or 200 μM DHEA. Cell number was determined 0, 24, 48, all 72 hours later using a Coulter counter (model Z; Coulter Electronics, Inc. Hialeah, Fla.). DHEA (AKZO, Basel, Switzerland) was dissolved in dimethyl sulfoxide, filter sterilized, and stored at −20° C. until use.

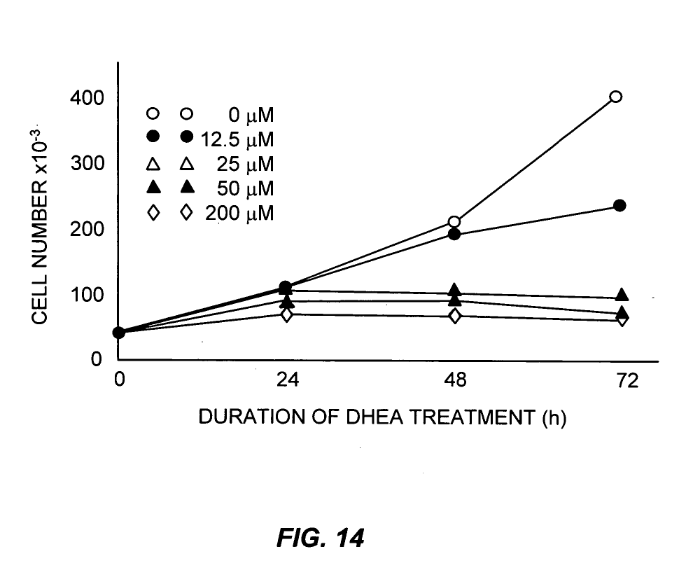

FIG. 14 illustrates the inhibition of growth for HT-29 cells by DHEA. Points refer to numbers of cells, and bars refer to SEM. Each data point was performed in quadruplicate, and the experiment was repeated three times. Where SEM bars are not apparent, SEM was smaller than symbol. Exposure to DHEA resulted in a reduced cell number compared to controls after 72 hours in 12.5 μM, 48 hours in 25 or 50 μM, and 24 hours in 200 μM DHEA, indicating that DHEA produced a time- and dose-dependent inhibition of growth.

DHEA Effect on Cell Cycle

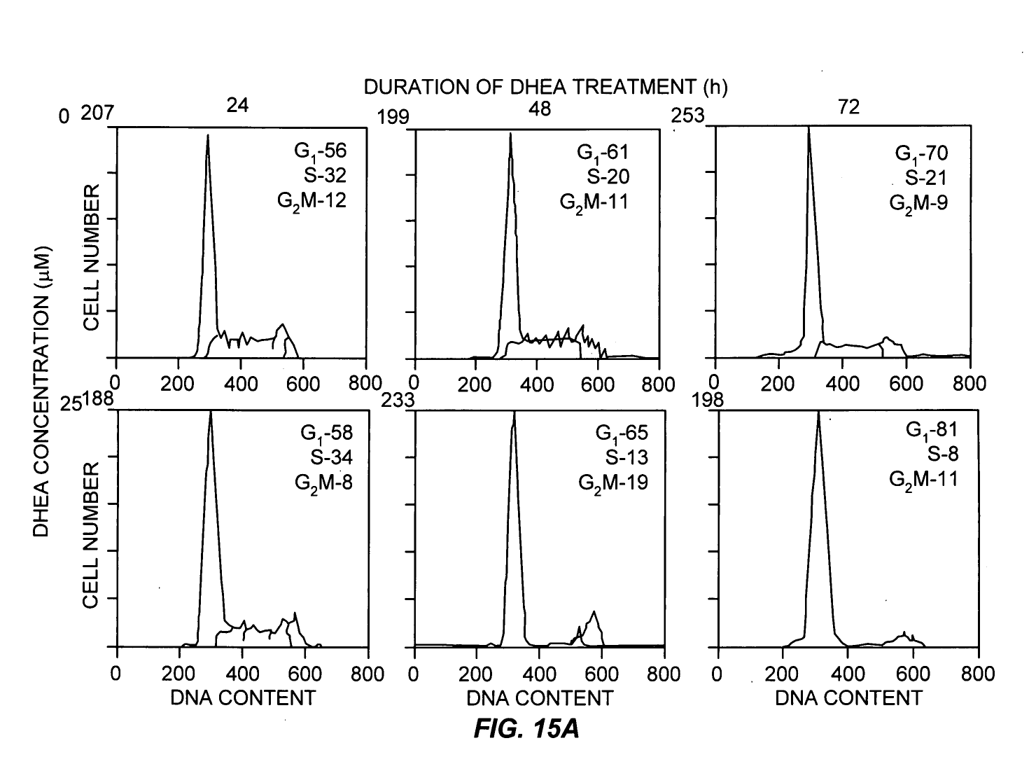

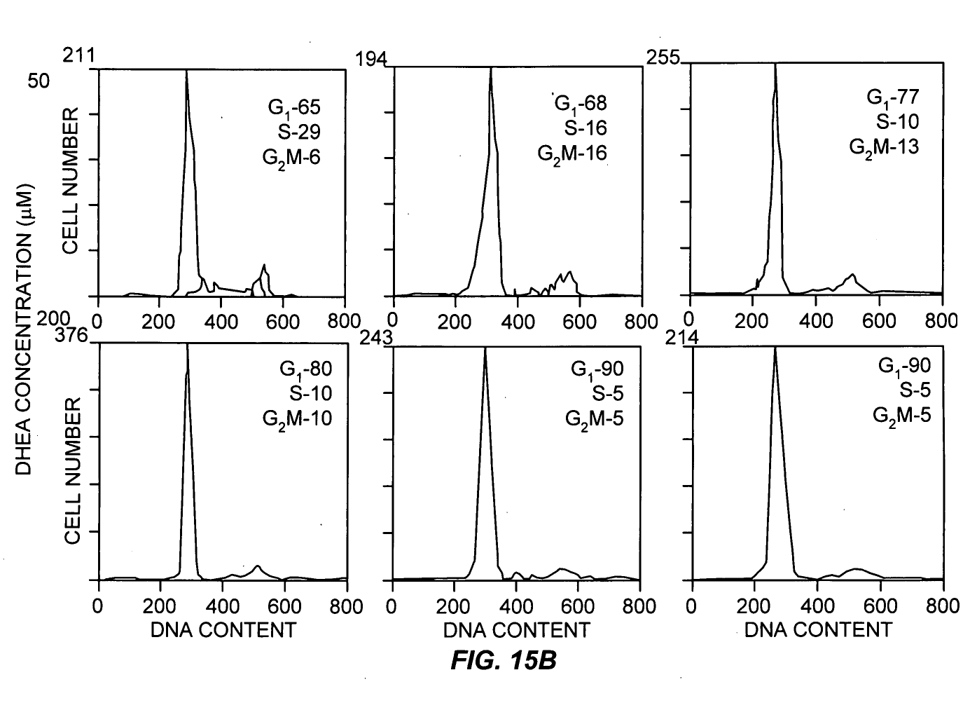

To examine the effects of DHEA on cell cycle distribution, HT-29 SF cells were plated (10⁵ cells/60 mm dish), and 48 hours later treated with 0, 25, 50, or 200 μM DHEA. FIG. 15 illustrates the effects of DHEA on cell cycle distribution in HT-29 SF cells. After 24, 48, and 72 hours, cells were harvested, fixed in ethanol, and stained with propidium iodide, and the DNA content/cell was determined by flow cytometric analysis. The percentage of cells in G₁, S, and G₂M phases was calculated using the Cellfit cell cycle analysis program. S phase is marked by a quadrangle for clarity. Representative histograms from duplicate determinations are shown. The experiment was repeated three times.

The cell cycle distribution in cultures treated with 25 or 50 μM DHEA was unchanged after the initial 24 hours. However, as the time of exposure to DHEA increased, the proportion of cells in S phase progressively decreased, and the percentage of cells in G₁, S and G₂M phases was calculated using the Cellfit cell cycle analysis program. S phase is marked by a quadrangle for clarity. Representative histograms from duplicate determinations are shown. The experiment was repeated three times.

The cell cycle distribution in cultures treated with 25 or 50 μM DHEA was unchanged after the initial 24 hours. However, as the time of exposure to DHEA increased, the proportion of cells in S phase progressively decreased and the percentage of cells in G₁ phase was increased after 72 hours. A transient increase in G₂M phase cells was apparent after 48 hours. Exposure to 200 μM DHEA produced a similar but more rapid increase in the percentage of cells in G₁ and a decreased proportion of cells in S phase after 24 hours, which continued through the treatment. This indicates that DHEA produced a G₁ block in HT-29 SF cells in a time- and dose dependent manner

### Example 10

### Reversal of DHEA-mediated Effect on Growth and Cell Cycle Reversal of DHEA-Mediated Growth Inhibition

Cells were plated as above, and after 2 days received either 0 or 25 μM DHEA-containing medium supplemented with mevalonic acid (“MVA”; mM) squalene (SQ; 80 μM), cholesterol (CH; 15 μg/ml), MVA plus CH, ribonucleosides (RN; uridine, cytidine, adenosine, and guanosine at final concentrations of 30 μM each), deoxyribonucleosides (DN; thymidine, deoxycytidine, deoxyadenosine and deoxyguanosine at final concentrations of 20 μM each). RN plus DN, or MVA plus CH plus RN, or medium that was not supplemented. All compounds were obtained from Sigma Chemical Co. (St. Louis, Mo.) Cholesterol was solubilized in ethanol immediately before use. RN and DN were used in maximal concentrations shown to have no effects on growth in the absence of DHEA.

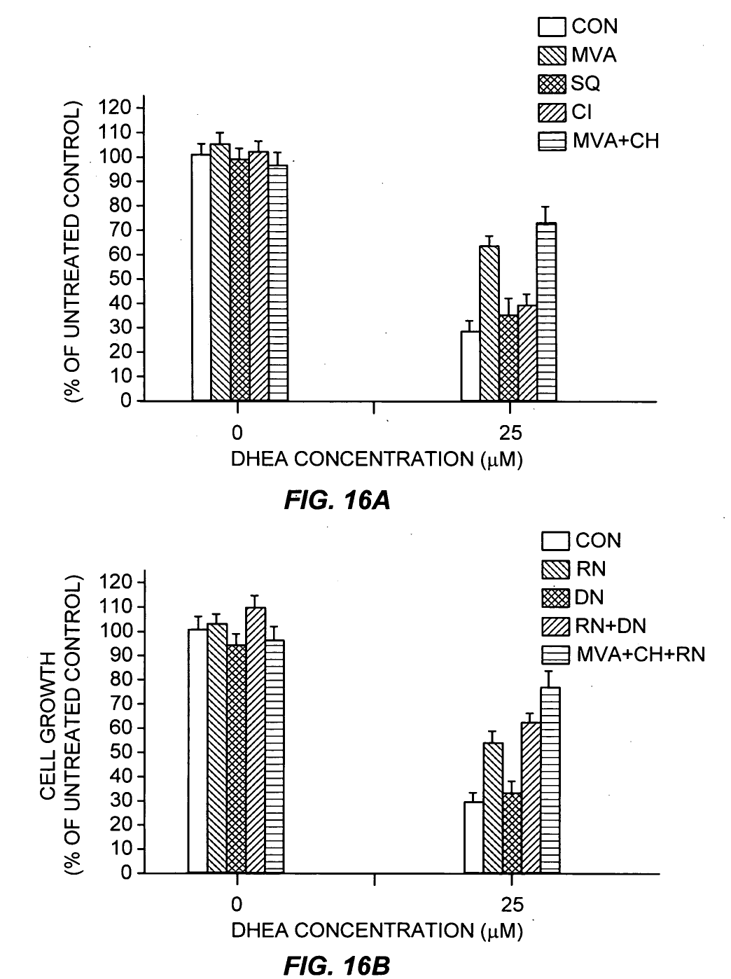

FIG. 16 illustrates the reversal of DHEA-induced growth inhibition in HT-29 SF cells. In A, the medium was supplemented with 2 μM MVA, 80 μM SQ, 15 μg/ml CH, or MVA plus CH (MVA+CH) or was not supplemented (CON). In B, the medium was supplemented with a mixture of RN containing uridine, cytidine, adenosine, and guanosine in final concentrations of 30 μM each; a mixture of DN containing thymidine, deoxycytidine, deoxyadenosine and deoxyguanosine in final concentrations of 20 μM each; RN plus DN (RN+DN); or MVA plus CH plus RN (MVA+CH+RN). Cell numbers were assessed before and after 48 hours of treatment, and culture growth was calculated as the increase in cell number during the 48 hour treatment period. Columns represent cell growth percentage of untreated controls; bars represent SEM. Increase in cell number in untreated controls was 173,370″6518. Each data point represents quadruplicate dishes from four independent experiments. Statistical analysis was performed using Student's t test κp<0.01; ψp<, 0.001; compared to treated controls. Note that supplements had little effect on culture growth in absence of DHEA.

Under these conditions, the DHEA-induced growth inhibition was partially overcome by addition of MVA as well as by addition of MVA plus CH. Addition of SQ or CH alone had no such effect. This suggest that the cytostatic activity of DHEA was in part mediated by depletion of endogenous mevalonate and subsequent inhibition of the biosynthesis of an early intermediate in the cholesterol pathway that is essential for cell growth. Furthermore, partial reconstitution of growth was found after addition of RN as well as after addition of RN plus DN but not after addition of DN, indicating that depietion of both mevalonate and nucleotide pools is involved in the growth-inhibitory action of DHEA. However, none of the reconstitution conditions including the combined addition of MVA, CH, and RN completely overcame the inhibitory action of DHEA, suggesting either cytotoxic effects or possibly that additional biochemical pathways are involved.

Reversal of DHEA Effect on Cell Cycle

HT-29 SF cells were treated with 25 FM DHEA in combination with a number of compounds, including MVA, CH, or RN, to test their ability to prevent the cell cycle-specific effects of DHEA. Cell cycle distribution was determined after 48 and 72 hours using flow cytometry.

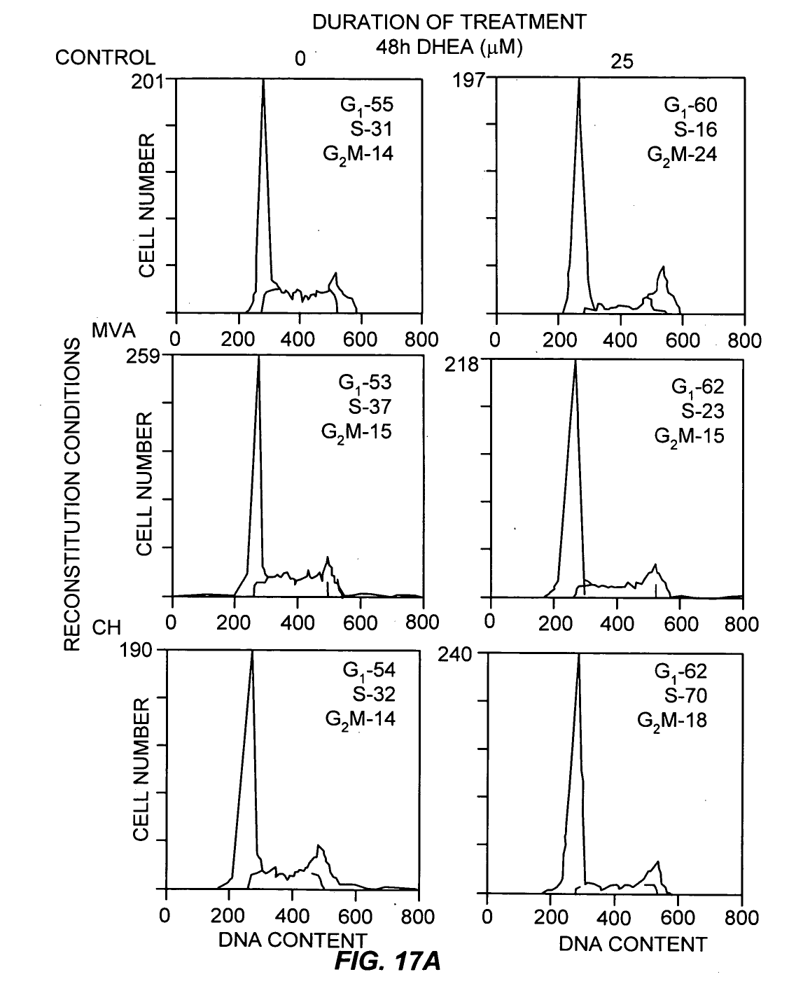

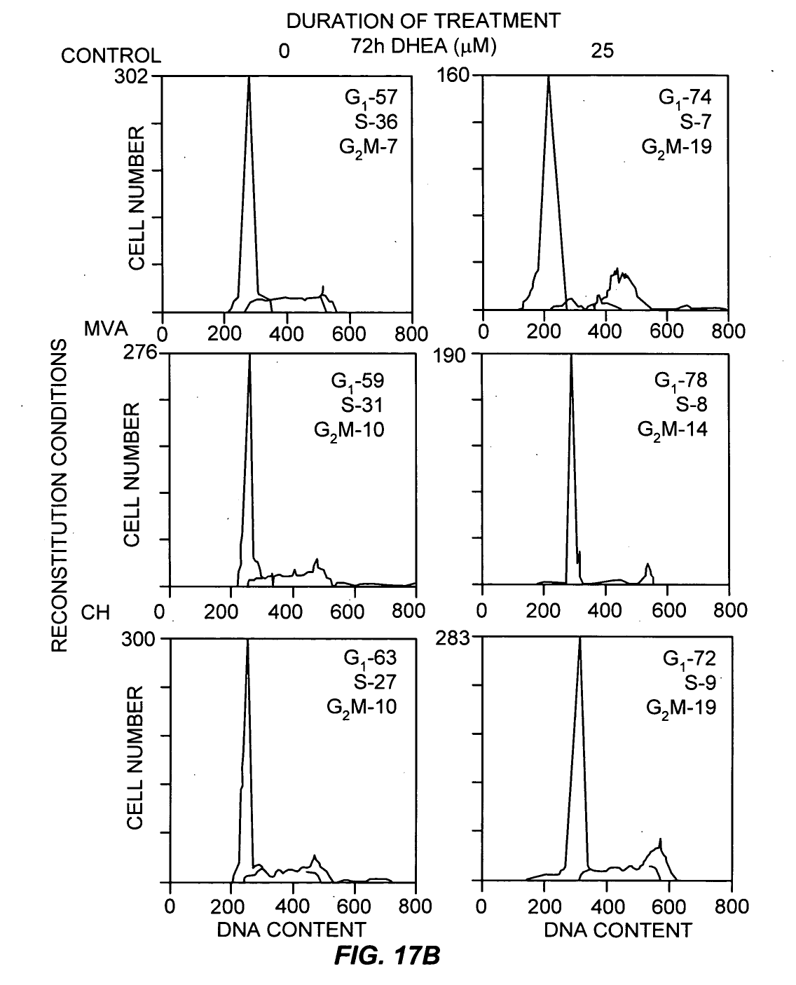

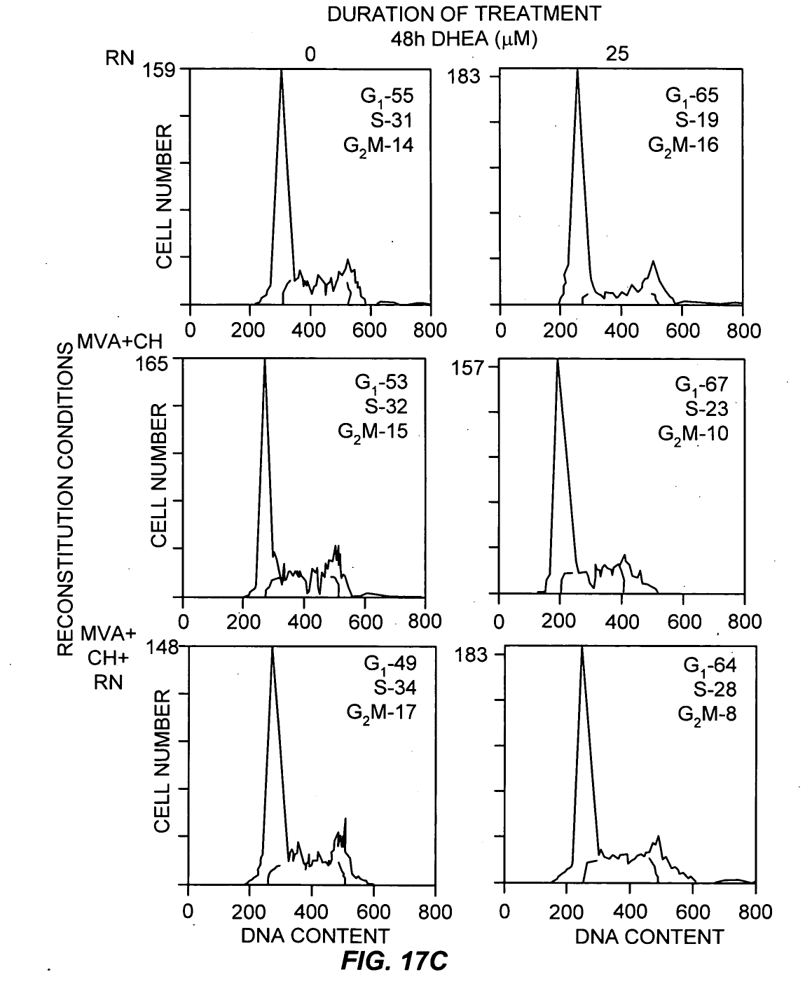

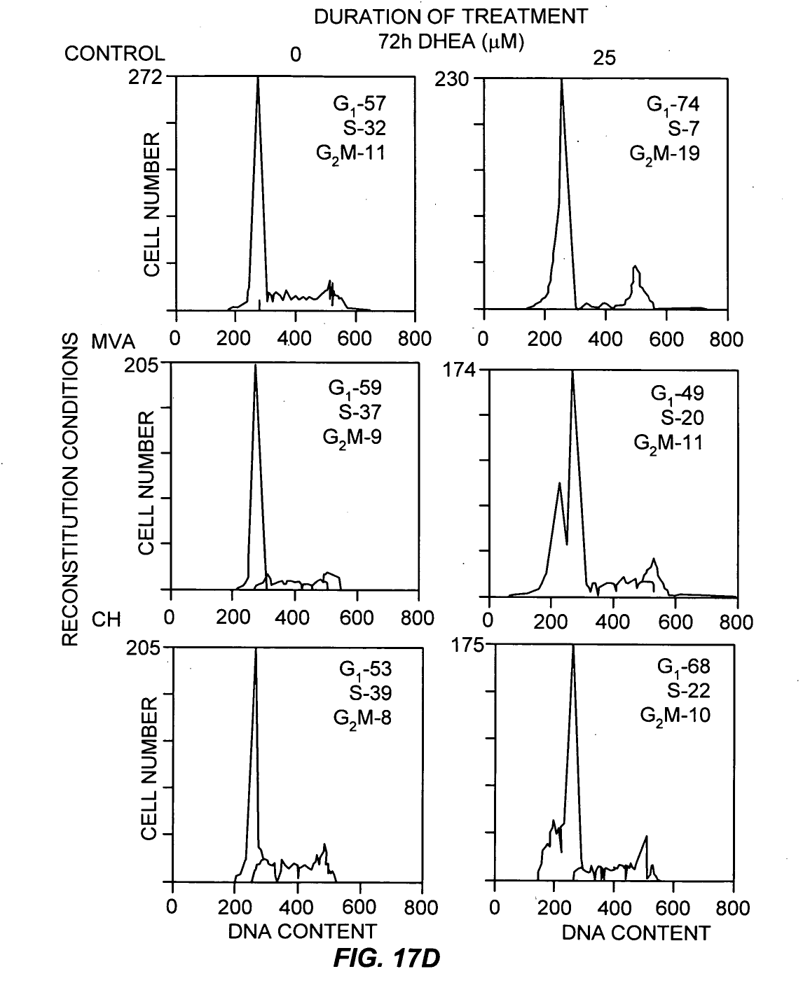

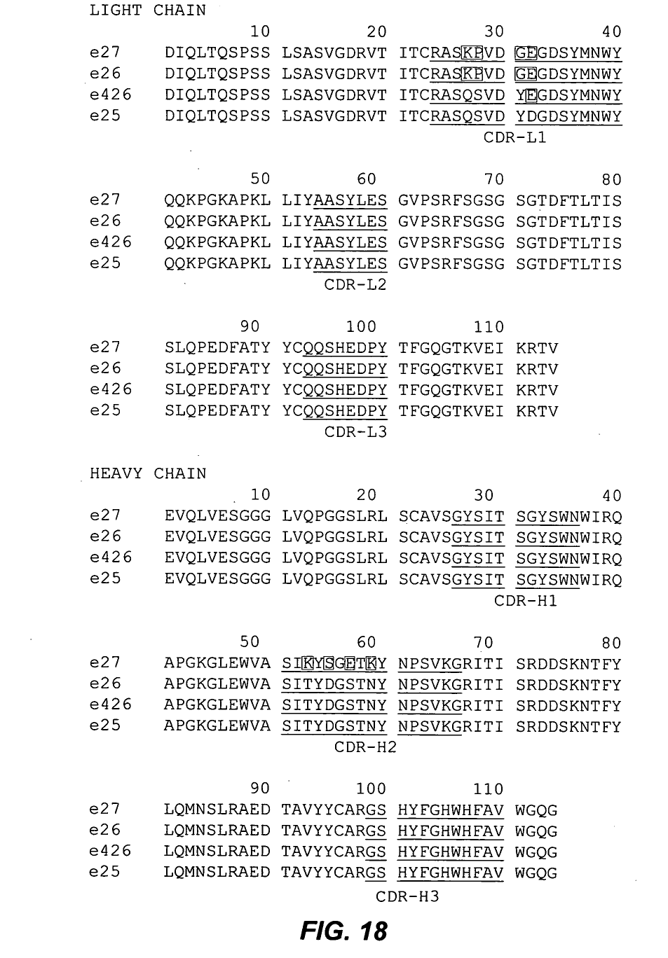

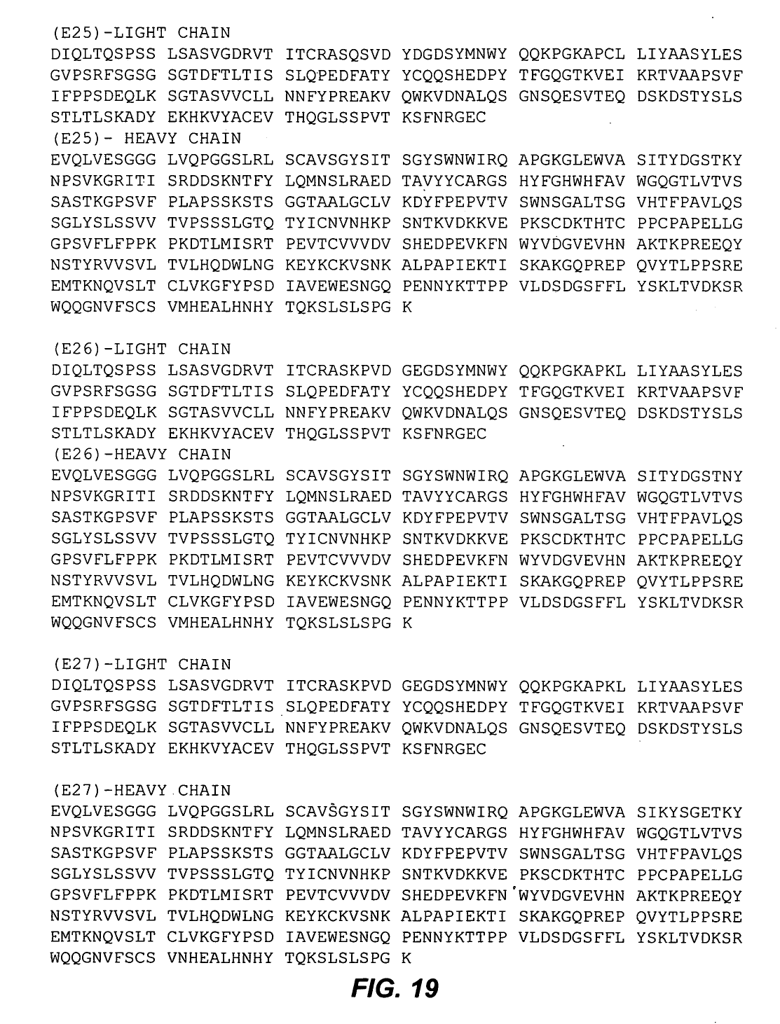

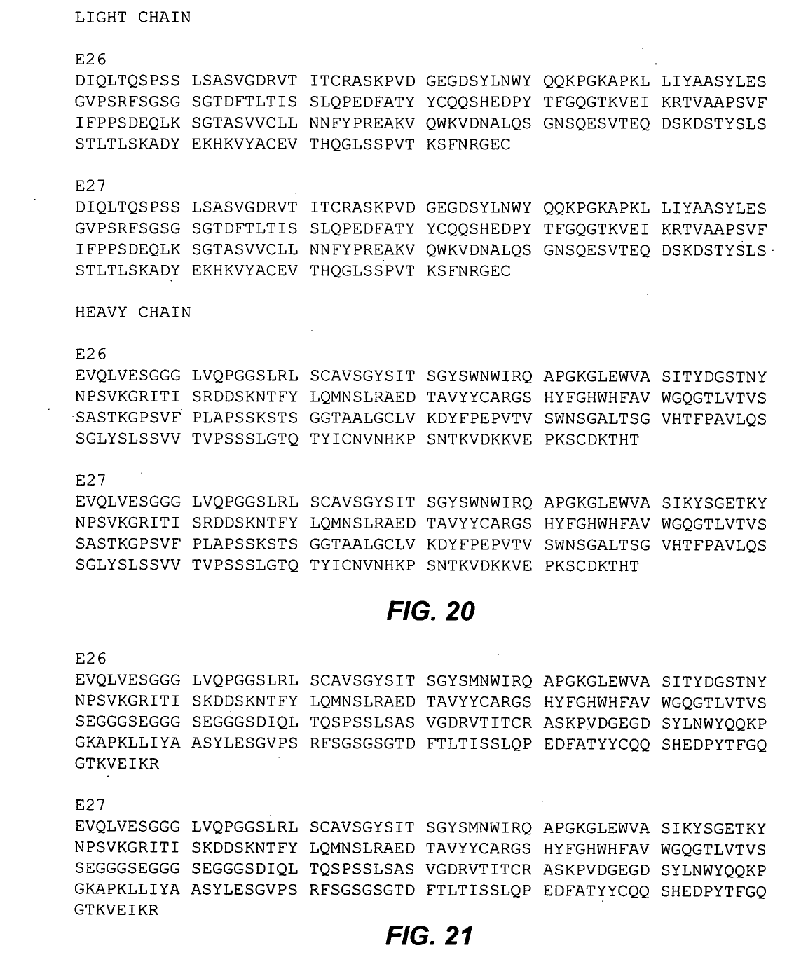

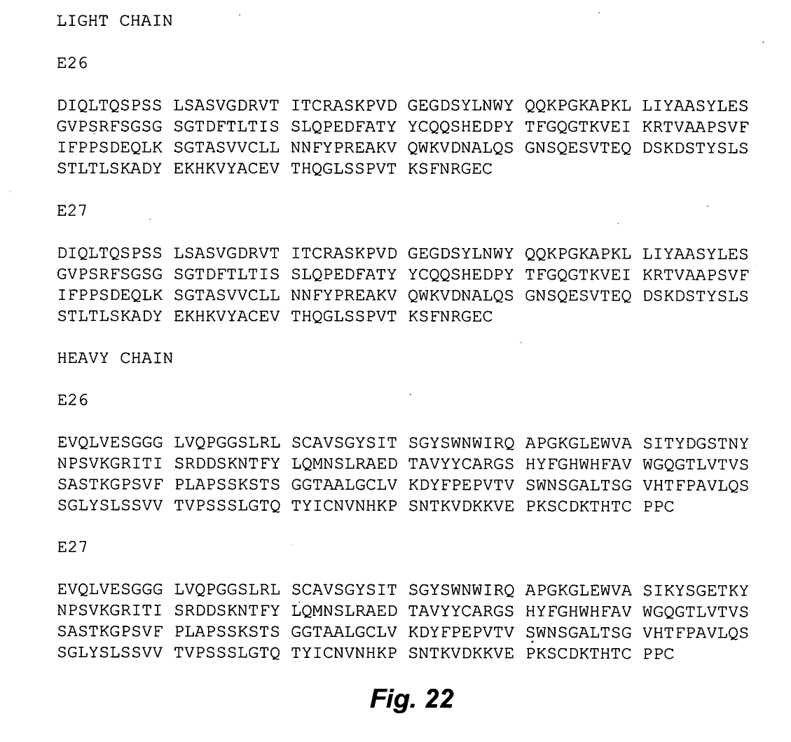

FIG. 17 illustrates reversal of DHEA-induced arrest in HT-29 SF cells. Cells were plated (10⁵ cells/60 mm dish) and 48 hours later treated with either 0 or 25 FM DHEA. The medium was supplemented with 2 FM MVA; 15 Fg/ml CH; a mixture of RN containing uridine, cytidine, adenosine, and guanosine in final concentrations of 30 FM; MVA plus CH (MVA+CH); or MVA plus CH plus RN (MVA+CH+RN) or was not supplemented. Cells were harvested after 48 or 72 hours, fixed in ethanol, and stained with propidium iodine, and the DNA content per cell was determined by flow cytometric analysis. The percentage of cells in G₁, S, and G₂M phases were calculated using the Cellfit cell cycle profile analysis program. S phase is marked by a quadrangle for clarity. Representative histograms from duplicative determinations are shown. The experiment was repeated two times. Note that supplements had little effect on cell cycle progression in the absence of DHEA.

With increasing exposure time, DHEA progressively reduced the proportion of cells in S phase. While inclusion of MVA partially prevented this effect in the initial 48 hours but not after 72 hours, the addition of MVA plus CH was also able to partially prevent S phase depletion at 72 hours, suggesting a requirement of both MVA and CH for cell progression during prolonged exposure. The addition of MVA, CH, and RN was apparently most effective at reconstitution but still did not restore the percentage of S phase cells to the value seen in untreated control cultures. CH or RN alone had very little effect at 48 hours and no effect at 72 hours. Morphologically, cells responded to DHEA by acquiring a rounded shape, which was prevented only by the addition of MVA to the culture medium. Some of the DNA histograms after 72 hours DHEA exposure in FIG. 4 also show the presence of a subpopulation of cells possessing apparently reduced DNA content. Since the HT-29 cell line is known to carry populations of cells containing varying numIbers of chromosomes (68-72; ATCC), this may represent a subset of cells that have segregated carrying fewer chromosomes.

### CONCLUSIONS

The Examples 9-10 above provide evidence that in vitro exposure of HT-29 SF human colonic adenocarcinoma cells to concentrations of DHEA known to deplete endogenous mevalonate results in growth inhibition and G₁ arrest and that addition of MVA to the culture medium in part prevents these effects. DHEA produced effects upon protein isoprenylation which were in many respects similar to those observed for specific 3-hydroxy-3-methyl-glutaryl-CoA reductase inhibitors such as lovastatin and compactin. Unlike direct inhibitors of mevalonate biosynthesis, however, DHEA mediates its effects upon cell cycle progression and cell growth in a pleiotropic manner involving ribo-and deoxyribonucleotide biosynthesis and possibly other factors as well.

### Example 11

### Metered Dose Inhaler

| Active Ingredient | Target per Actuation | |
|---|---|---|
| Omalizumab | 10.0 | mg |
| DHEA | 400 | mg |
| Stabilizer | 5.0 | μg |
| Trichlorofluoromethane | 23.70 | mg |
| Dichlorodifluoromethane | 61.25 | mg |

### Example 12

### Metered Dose Inhaler

| Active Ingredient | Target per Actuation | |
|---|---|---|
| Omalizumab | 10.0 | mg |
| DHEA-S | 400 | mg |
| Stabilizer | 7.5 | μg |
| Trichlorofluoromethane | 23.67 | mg |
| Dichlorodifluoromethane | 61.25 | mg |

In the following Examples 13 and 14, the first and second active agents are micronized and bulk blended with lactose in the proportions given above. The blend is filled into hard gelatin capsules or cartridges or into specifically constructed double foil blister packs (Rotadisks blister packs, Glaxo®) to be administered by an inhaler such as the Rotahaler inhaler (Glaxo®) or in the case of the blister packs with the Diskhaler inhaler (Glaxo®).

### Example 13

### Metered Dose Dry Powder Formulation

| Active Ingredient | /cartridge or blister | |
|---|---|---|
| Omalizumab | 10.0 | mg |
| DHEA | 1.00 | mg |
| Lactose Ph. Eur. | to 12.5 or 25.0 | mg |

### Example 14

### Metered Dose Dry Powder Formulation

| Active Ingredient | /cartridge or blister | |
|---|---|---|
| Omalizumab | 10.0 | mg |
| DHEA-S | 1.00 | mg |
| Lactose Ph. Eur. | to 12.5 or 25.0 | mg |

Although the invention has been described with reference to the presently preferred embodiments, it should be understood that various modifications can be made without departing from the spirit of the invention.

All publications, patents, and patent applications, and web sites are herein incorporated by reference in their entirety to the same extent as if each individual publication, patent, or patent application, was specifically and individually indicated to be incorporated by reference in its entirety.

1. A pharmaceutical composition, comprising a pharmaceutically or veterinarily acceptable carrier, a first active agent and a second active agent effective to treat asthma, chronic obstructive pulmonary disease, or a respiratory or lung disease,
(a) the first active agent is at least one of a non-glucocorticoid steroid selected from a non-glucocorticoid steroid having the chemical formula 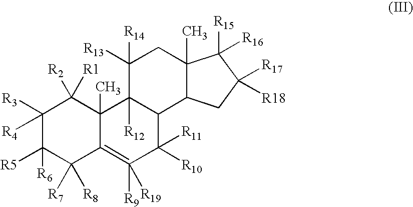
and a non-glucocorticoid steroid of the chemical formula 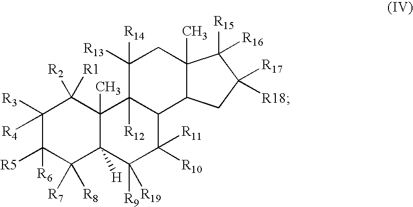
wherein R1, R2, R3, R4, R5, R7, R8, R9, R10, R12, R13, R14 and R19 are independently H, OR, halogen, (C1-C10) alkyl or (C1-C10) alkoxy, R5 and R11 are independently OH, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO2R20, —OPOR2OR21 or (C1-C10) alky, R5 and R6 taken together are ═O, R10 and R11 taken together are ═O; R15 is (1) H, halogen, (C1-C10) alkyl, or (C1-C10) alkoxy when R16 is —C(O)OR22, (2) H, halogen, OH or (C1-C10) alkyl when R16 is halogen, OH or (C1-C10) alkyl, (3) H, halogen, (C1-C10) alkyl, (C1-C10) alkenyl, (C1-C10) alkynyl, formyl, (C1-C10) alkanoyl or epoxy when R16 is OH, (4) OR, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO2R20 or —OPOR2OR21 when R16 is H, or R15 and R16 taken together are ═O; R17 and R18 are independently (1) H, —OH, halogen, (C1-C10) alkyl or —(C1-C10) alkoxy when R6 is H OR, halogen. (C1-C10) alkyl or —C(O)OR22, (2) H, (C1-C10 alkyl).amino, ((C1-C10) alkyl)ₙ amino-(C1-C10) alkyl, (C1-C10) alkoxy, hydroxy-(C1-C10) alkyl, (C1-C10) alkoxy-(C1-C10) alkyl, (halogen)m (C1-C10) alkyl, (C1-C10) alkanoyl, formyl, (C1-C10) carbalkoxy or (C1-C10) alkanoyloxy when R15 and R16 taken together are =0, (3) R17 and R18 taken together are ═O; (4) R17 or R18 taken together with the carbon to which they are attached form a 3-6 member ring containing 0 or 1 oxygen atom; or (5) R15 and R17 taken together with the carbons to which they are attached form an epoxide ring; R20 and R21 are independently OH, pharmaceutically acceptable ester or pharmaceutically acceptable ether; R22 is H, (halogen)ₘ (C1-C10) alkyl or (C1-C10) alkyl; n is 0, 1 or 2; and m is 1, 2 or 3; or pharmaceutically or veterinarily acceptable salts thereof; and
(b) the second active agent is an anti-IgE antibody.

2. The pharmaceutical composition of claim 1 wherein said first active agent is a non-glucocorticoid steroid having the chemical formula 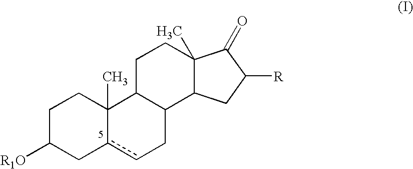
wherein the broken line represents a single or a double bond; R is hydrogen or a halogen; the H at position 5 is present in the alpha or beta configuration or the compound of chemical formula I comprises a racemic mixture of both configurations; and R¹ is hydrogen or a multivalent inorganic or organic dicarboxylic acid covalently bound to the compound.

3. The pharmaceutical composition of claim 1, wherein the first active agent is a non-glucocorticoid steroid having the chemical formula (I), wherein said multivalent organic dicarboxylic acid is SO₂₀M, phosphate or carbonate, wherein M comprises a counterion, wherein said counterion is H, sodium, potassium, magnesium, aluminum, zinc, calcium, lithium, ammonium, amine, arginine, lysine, histidine, triethylamine, ethanolamine, choline, triethanoaamine, procaine, benzathine, tromethanine, pyrrolidine, piperazine, diethylamine, sulfatide 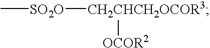
or phosphatide 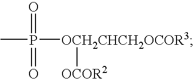
wherein R² and R³, which are the same or different, and are straight or branched (C₁-C₁₄) alkyl or glucuronide

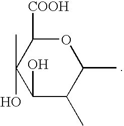

4. The pharmaceutical composition of claim 3, wherein said first active agent is dehydroepiandrosterone.

5. The pharmaceutical composition of claim 3, wherein said first active agent is dehydroepiandrosterone-sulfate.

6. The pharmaceutical composition of claim 1, wherein said anti-IgE antibody is E-25 (omalizumab).

7. The pharmaceutical composition of claim 1, further comprising a ubiquinone or pharmaceutically or veterinarily acceptable salt thereof, wherein the ubiquinone has the chemical formula 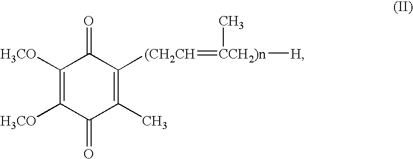
wherein n is 1 to 12.

8. The pharmaceutical composition of claim 1, wherein the pharmaceutical composition comprises particles of inhalable or respirable size.

9. The pharmaceutical composition of claim 8, wherein the particles are about 0.01 μm to about 10 μm in size.

10. The pharmaceutical composition of claim 8, wherein the particles are about 10 μm to about 100 μm in size.

11. A kit comprising a delivery device and the pharmaceutical composition of claim 1.

12. The kit of claim 11, wherein the delivery device is an aerosol generator or spray generator.

13. The kit of claim 12, wherein the aerosol generator comprises an inhaler.

14. The kit of claim 13, wherein the inhaler delivers individual pre-metered doses of the formulation

15. The kit of claim 13, wherein the inhaler comprises a nebulizer or insufflator.

16. A method for reducing the probability of or treating asthma in a subject, comprising administering to a subject in need of such treatment a prophylactically or therapeutically effective amount of the pharmaceutical composition of claim 1.

17. A method for reducing the probability of or treating of chronic obstructive pulmonary disease in a subject, comprising administering to a subject in need of such treatment a prophylactically or therapeutically effective amount of the pharmaceutical composition of claim 1.

18. A method for treatment of respiratory, lung or malignant disorder or condition, or for reducing levels of; or sensitivity to, adenosine or adenosine receptors in a subject, comprising administering to a subject in need of such treatment a prophylactically or therapeutically effective amount of the pharmaceutical composition of claim 1.

19. The method of claim 18, wherein the disorder or condition comprises asthma, chronic obstructive pulmonary disease (COPD), cystic fibrosis (CF), dyspnea, emphysema, wheezing, pulmonary hypertension, pulmonary fibrosis, hyper-responsive airways, increased adenosine or adenosine receptor levels, adenosine hyper-sensitivity, infectious diseases, pulmonary bronchoconstriction, respiratory tract inflammation or allergies, lung surfactant or ubiquinone depletion, chronic bronchitis, bronchoconstriction, difficult breathing, impeded or obstructed lung airways, adenosine test for cardiac function, pulmonary vasoconstriction, impeded respiration, Acute Respiratory Distress Syndrome (ARDS), administration of adenosine or adenosine level increasing drugs, infantile Respiratory Distress Syndrome (infantile RDS), pain, allergic rhinitis, cancer, or chronic bronchitis.
